SEQUENCE LISTING (9 sequences)

SEQ ID NO: 1 - DNA, 45 bp, artificial sequence
  artificial intron sequences derived from nematode genes
gtacttgagatccttaaacgcagtcgaaaattggtaattttacag

SEQ ID NO: 2 - DNA, 45 bp, artificial sequence
  artificial intron sequences derived from nematode genes
gtaagttcctccactagaaatatcaggtgctataattgtgttcag

SEQ ID NO: 3 - DNA, 48 bp, artificial sequence
  artificial intron sequences derived from nematode genes
gtgagttattataatttttttgatcacaacgattattttaattttcag

SEQ ID NO: 4 - DNA, 51 bp, artificial sequence
  artificial intron sequences derived from nematode genes
gtgagtgattttaaacattatctgtacttaaattataaattctctattcag

SEQ ID NO: 5 - DNA, 48 bp, artificial sequence
  artificial intron sequence derived from nematode genes
gtaaataattatacattcgatgataaatttatgcgtactatttttcag

SEQ ID NO: 6 - DNA, 51 bp, artificial sequence
  artificial intron sequence derived from nematode genes
gttaaatgtacaaacaactatttgaaagattttctcacccgattttttcag

SEQ ID NO: 7 - DNA, 20 bp, artificial sequence
  sgRNA targeting sequence
acaacaacatgactaaggac

SEQ ID NO: 8 - DNA, 20 bp, artificial sequence
  sgRNA targeting sequence
gaaactttcagaatgcagga

SEQ ID NO: 9 - DNA, 108 bp, artificial sequence
  oligonucleotide DNA
cgagacctgtcaacaggaacaacaacatgactaagtaaataaataaaccccagaagtcctccagtccctcgagggaagggttcccatgcacttggtcgatttgcacct

(19) United States
(12) Patent Application Publication
(10) Pub. No.: US 2023/0146102 A1
(43) Pub. Date: May 11, 2023

(54) MONOGENIC OR POLYGENIC DISEASE MODEL ORGANISMS HUMANIZED WITH TWO OR MORE GENES

(71) Applicant: NemaMetrix, Inc, Eugene, OR (US)

(72) Inventors: Christopher E. Hopkins, Salt lake City, UT (US); Trisha J. Brock, Eugene, OR (US); Richard Fekete, Eugene, OR (US); Kathryn McCormick, Eugene, OR (US)

(73) Assignee: NemaMetrix, Inc, Eugene, OR (US)

(21) Appl. No.: 17/433,221

(22) PCT Filed: Feb. 21, 2020
(86) PCT No.: PCT/US2020/019308

§ 371 Date: Aug. 23, 2021

Related U.S. Application Data
(63) Continuation of application No. 16/281,988, filed on Feb. 21, 2019, now Pat. No. 11,477,970.
(60) Provisional application No. 62/821,377, filed on Mar. 20, 2019.
(60) Provisional application No. 62/633,590, filed on Feb. 21, 2018.
(60) Provisional application No. 62/653,092, filed on Apr. 5, 2018.

(51) Int. Cl. A01K 67/033; A61K 49/00; C12N 15/85; G01N 33/50

(52) U.S. Cl. CPC A01K 67/0336; A61K 49/0008; C12N 15/8509; G01N 33/5085; A01K 2207/15; A01K 2217/077; A01K 2227/703; A01K 2267/0318; A01K 2267/0393; C12N 2015/8536; C12N 2015/859; G01N 2333/43534

(57) ABSTRACT

The present disclosure provides transgenic non-human animal (e.g., nematode) systems for assessing heterologous polygenic or monogenic phenotypes, their variants and drug discovery. The transgenic non-human animals (e.g., nematodes) contain a first heterologous polypeptide coding sequence and a second heterologous polypeptide coding sequence (a plurality of heterologous polypeptide coding sequences), wherein the first and second heterologous polypeptide coding sequences are integrated into the host animal genome, and wherein expression of the first and second heterologous polypeptide coding sequence contribute to the heterologous phenotype. The plurality of heterologous polypeptide coding sequences are interrelated wherein their expression products, directly or indirectly, contribute or lead to an observable phenotype.

### CROSS-REFERENCE TO RELATED APPLICATIONS

This application claims the benefit of U.S. Provisional Patent Application No. 62/821,377, filed on 20 Mar. 2019, the content of which is incorporated herein by reference in its entirety.

This application claims priority to pending U.S. Ser. No. 16/281,988, filed on 21 Feb. 2019, and to pending PCT/US19/19027, filed 21 Feb. 2019, the contents of which are each incorporated herein by reference in their entirety.

### SEQUENCE LISTING

The instant application contains a Sequence Listing which has been submitted electronically in ASCII format via EFS-Web and hereby incorporated by reference in its entirety. Said ASCII copy, created on 21 Feb. 2020, is named NEMA013PCT_ST25.TXT and is 2384 bytes in size.

### FIELD OF THE DISCLOSURE

This application pertains generally to transgenic animals comprising two or more heterologous polypeptide coding sequences, wherein expression of the heterologous polypeptide coding sequence product contributes to the same heterologous phenotype; and their use in assessing monogenic or polygenic diseases and gene variants thereof.

### BACKGROUND OF THE DISCLOSURE

Clinical genomics is revealing genetic variation occurs at high prevalence in the human population. Accumulated genomic data reveals each person has about 500 sequence variants that create missense or indel mutations in the coding regions of their genome (Jansen I et al. Establishing the role of rare coding variants in known Parkinson's disease risk loci. Neurobiol Aging. 2017 November; 59:220.e11-220.e18). With estimates as high as 30% of the genes in the human genome being involved in disease biology (Hegde M et al. Development and Validation of Clinical Whole-Exome and Whole-Genome Sequencing for Detection of Germline Variants in Inherited Disease. Arch Pathol Lab Med. 2017 June; 141(6):798-805.), any one individual harbors over 100 codon-changing variations in their important “disease” genes. Surprisingly, frameshifting indels with a high likelihood of pathogenicity account for only 7% of these variants. As a result, there remains a significant number of questionable alleles that are part of the background of anyone's personal genome. The challenge to the physician is to determine if a suspect allele is contributing to the disease as a pathogenic variant or if the clinical variant is not consequential and can be classified as a benign variant. For many of the genetic differences seen in a patient's genome, the benign or pathogenic status remains undefined and the variant is a Variant of Uncertain Significance (VUS). As a result, variant interpretation is the major bottleneck now that large scale sequencing is increasingly being used in clinical settings.

Genome wide association studies (GWAS) reveal multiple genes are involved in many types of disease. For instance, a study of the polygenic genetic architecture of schizophrenia identified more than 10% of genome (2725 candidate genes) may be acting as risk factors for disease (Lee et al “Estimating the proportion of variation in susceptibility to schizophrenia captured by common SNPs.” Nat Genet. 2012 Feb. 19; 44(3):247-50). Another SNP-based GWAS in epilepsy identified 16 genetic regions containing 21 epilepsy target genes as highly-associated with adult onset disease (Abou-Khalil et al. “Genome-wide mega-analysis identifies 16 loci and highlights diverse biological mechanisms in the common epilepsies” Nat Commun. 2018 Dec. 10; 9(1):5269). Yet a challenge of GWAS is to identify the molecular nature of the polygenic drivers of disease. Most SNPs in an association cluster occur in non-coding regions. For the rare GWAS SNP that occurs in coding segments they tend to be in non-conserved regions. As a result, they are rarely the molecular cause of the disease risk factor. Instead it is a rare minor allele at a nearby SNP located within a low to non-recombination interval on the same strand as one of the GWAS high frequency SNPs. Since thousands of rare SNPs can fall into this category it becomes challenging and tedious to identify the molecular cause of a polygenic contribution to disease. Systems are needed for looking at the additive effects on gene disfunction for a set of rare alleles distributed across more than one loci.

A significant proportion of clinical variants seen in patients with genetic disease are caused by missense changes resulting in altered amino acid usage. Unlike the rarer frameshift and stop-codon mutations and some intra-/inter-genic variants, the functional consequence of missense amino acid changes can remain elusive. Change of function due to missense can result in partial loss of gene activities or gain-of-function changes that are highly pathogenic. There is an emergent need for the functional analysis of variant pathogenicity that occurs as a result of these amino acid changes.

A variety of technologies from bioinformatics to biochemical assays can be deployed to assess functional consequence of missense changes. Yet the most reliable are the in vivo systems. Most commonly used are cell culture assays that translate to animal model studies. The lack of intact animal biology occurring in cell culture systems renders this technique intractable to many transcellular pathogenicities. As a result, transgenic animal models are favored for capturing the nuances of intra- and inter-cellular pathogenicity in native contexts.

Transgenic mice are the traditional animal model for probing functional consequence of genomic variation. Yet their high expense and low throughput leave their use as intractable to address the 100,000,000's of coding altering variants predicted to occur in human populations. Many groups are now focusing on using alternative model organisms (Zebrafish, Drosophila and C. elegans) as a more affordable and timely approach to assessing variant specific effects on gene function, for example, the Undiagnosed Disease Network). Yet current design compositions and features of the transgenics used in these studies are not as efficient or appropriate as they could be for accurate assessment of variant function.

As one of the five classical model organisms for genetic studies (worm, fly, yeast, zebrafish and mice) the C. elegans nematode worm has a unique set of attributes that make it highly optimal for high-throughput clinical variant phenotyping. At the genetic level, the C. elegans nematode rivals the Drosophila fly for having orthologs to 80% of human disease genes, wherein 6460 genes detected in ClinVar Miner database as human disease genes were queried for homologs using the DIOPT database (Hu Y et al. An integrative approach to ortholog prediction for disease-focused and other functional studies. BMC Bioinformatics. 2011 Aug 31; 12:357). Of the multicellular models, the C. elegans animal model has the fastest life cycle (3 days). It has optical transparency for easy tissue and organ system expression observation. Finally, in a unique advantage of interpretability, the C. elegans animals are easy to breed as self-fertilizing hermaphrodites, which allow rapid population expansion of nearly identical animals with very minimal polymorphism load in the genetic background. This allows transgenesis and subsequent population phenotyping to be performed in a matter of a few weeks instead of years.

Transgenic C. elegans are optimal for drug screening capacity. Of the five animal models, only yeast provides higher diversity screening per meter of bench space in comparison to C. elegans. Yet, yeast exist in a single cellular context and it becomes challenging to accurately model human biology where variant function (or disfunction) operates in a 3-dimensional tissue-based architecture. The advent of iPSC (Csöbönyeiová, M et al. Recent Advances in iPSC Technologies Involving Cardiovascular and Neurodegenerative Disease Modeling. General Physiology and Biophysics 35, no. 1 (January 2016): 1-12) and organoid (Breslin S and O'Driscoll L. Three-Dimensional Cell Culture: The Missing Link in Drug Discovery. Drug Discovery Today 18, no. 5-6 (March 2013): 240-49) technologies bring more biological-context relevance, yet they remain undemonstrated for capacity to deploy in robust high-throughput formats. The C. elegans animal model, on the other hand, is robust and fast for high density screens of biological alterations. For instance, a recent screen for SKN-1 inhibitors as anthelmintic therapeutics found promising hits in few weeks screen of 340,000 compounds (Leung C K et al. An ultra high-throughput, whole-animal screen for small molecule modulators of a specific genetic pathway in Caenorhabditis elegans. PLoS One. 2013 Apr 29; 8 (4): e62166). Many other groups have used transgenic C. elegans for medium- to high-throughput drug discovery (Artal-Sanz M et al. Caenorhabditis elegans: a versatile platform for drug discovery. Biotechnol J. 2006 Dec.; 1(12):1405-18; O'Reilly L P et al. C. elegans in high-throughput drug discovery. Adv Drug Deliv Rev. 2014 April; 69-70:247-53; Xiong H et al. An enhanced C. elegans based platform for toxicity assessment. Sci Rep. 2017 Aug 29; 7(1):9839; Kim W et al. An update on the use of C. elegans for preclinical drug discovery: screening and identifying anti-infective drugs. Expert Opin Drug Discov. 2017 Jun.; 12(6):625-633; and, Kim H et al. A co-CRISPR strategy for efficient genome editing in Caenorhabditis elegans. Genetics. 2014 August; 197(4):1069-80).

C. elegans are a microscopic organism, with intact nervous system capable of learned behavior, where the animal can pack into 96 well, 384 well and even 1536 well assays (Leung, C. K., Deonarine, A., Strange, K. & Choe, K. P. High-throughput Screening and Biosensing with Fluorescent C. elegans Strains. J Vis Exp (2011)). It has complex tissue structure (nervous system, muscles, germ line, intestine, mouth-like pharynx, periodic excretion through anal sphincter, macrophage-like celomocytes, and a tough skin-like hypodermis). As a result, the C. elegans nematode provides complex tissue biology in an intact, easy-to-culture animal model.

Zebrafish have developed into a popular animal model platform for drug discovery with a fast-growing conference support (Zebrafish Disease Modeling Society) now in its 13^{th} year. Advantages of the use of zebrafish as an animal model are its inclusion in the vertebrate phylum which results in a high degree of homologous gene structures and organ systems in relation to humans. Breeds of zebrafish are available with high transparency (e.g. CASPER) which enable direct in vivo monitoring of gene activity and organ variability in live animals. Like the liquid format used in C. elegans, animal growth and handling of zebrafish is easily automated with a variety of fluidic systems.

Current variant modeling systems in zebrafish, C. elegans, and other animals are predominantly done as site directed mutagenesis to insert a variant at the native ortholog locus. Only a few groups have tried expression of human transgenes in these animal models to varying levels of success. A simple and robust approach to create ideal transgenic compositions is lacking. As a result, there remains a need for a ubiquitous transgenics platform that can be used to assess function of broad categories of clinical variants, and their interaction with expression of wild-type genes in vivo, and screen for drug discovery in the treatment of pathogenic clinical variants. Moreover, there remains a need for looking at the additive effects on gene disfunction for a set of rare alleles distributed across more than one loci.

Herein we provide an animal model transgenic platform wherein the animal model configuration frequently has the animal's ortholog replaced by a chimeric heterologous transgene, such as human disease exon coding sequences paired with a host animal (e.g. nematode) intron sequences, that can be used to increase understanding of individual variants (clinical and biological) as well as their interaction or additive effects with other variants or wild-type sequences that contribute to a particular disease. Furthermore, the resulting transgenic animal systems can be used to provide highly-personalized (variant-specific) discovery of therapeutic approaches.

### SUMMARY OF THE INVENTION

Herein are provided transgenic non-human animals systems for assessing a heterologous polygenic or monogenic phenotype and methods thereof. In embodiments, the non-human animal is a nematode or zebrafish. In embodiments, a transgenic nematode system comprises a host nematode comprising and expressing a first heterologous polypeptide coding sequence and a second heterologous polypeptide coding sequence, wherein the first and second heterologous polypeptide coding sequences are integrated into the host nematode genome, and wherein expression of the first and second heterologous polypeptide coding sequences contribute to the heterologous phenotype. The first and second heterologous polypeptide coding sequence(s) are interrelated in that their expression contributes to the same phenotype or trait. That phenotype may be a particular disease, such as a neurodegenerative disease.

In embodiments, the host animal further comprises and expresses one or more additional heterologous polypeptide coding sequence that contribute to the heterologous phenotype. In embodiments, the host nematode comprises and expresses 2 to 15 heterologous polypeptide coding sequences; or 3 to 15 heterologous polypeptide coding sequences. In certain embodiments, the one or more additional heterologous polypeptide coding sequence(s) comprises one or more mutations in exon coding sequences of the heterologous polypeptide coding sequence as compared to a wildtype reference sequence resulting in at least one amino acid change when the one or more additional heterologous polypeptide coding sequence is expressed.

In embodiments, the heterologous polypeptide coding sequence replaces the nematode ortholog using gene swap techniques involving removing the native coding sequence of the host nematode ortholog and replacing with modified cDNA coding sequence from a heterologous polypeptide sequence.

The choice of introduced transgene sequence can vary widely but in one embodiment the sequence is a modified cDNA coding sequence from any eukaryotic organism. In embodiments, Applicants found that using modified intron sequences from a highly expressed gene of the host nematode, paired with or interspersed with the heterologous exon coding sequences—a chimeric heterologous polypeptide coding sequence—improved expression of the heterologous polypeptide coding sequence in the host nematode. (See U.S. Ser. No. 16/281,988, filed 21 Feb. 2019, incorporated in its entirety herein by reference). Accordingly, in certain embodiments, at least one of the first heterologous polypeptide coding sequence or the second heterologous polypeptide coding sequence is a chimeric heterologous polypeptide coding sequence comprising heterologous exon coding sequences interspersed with artificial host nematode intron sequences optimized for expression in the host nematode. In further embodiments, each of the first and second heterologous polypeptide coding sequence is individually a chimeric heterologous polypeptide coding sequence comprising heterologous exon coding sequences interspersed with artificial host nematode intron sequences optimized for expression in the host nematode.

In embodiments provided herein is a transgenic nematode comprising and expressing a first heterologous polypeptide coding sequence and a second heterologous polypeptide coding sequence, wherein the host nematode comprises a chimeric heterologous polypeptide coding sequence comprising heterologous exon coding sequences interspersed with artificial host nematode intron sequences optimized for expression in the host nematode selected from SEQ ID NO: 1, 2, 3, 4, 5 or 6. In addition to introduction of artificial host intron sequences into the cDNA sequence from the heterologous polypeptide coding sequence, the chimeric heterologous polypeptide coding sequence may be optimized for expression in the host nematode wherein the heterologous polypeptide coding sequence is codon optimized for the host nematode and aberrant splice donor and/or acceptor sites removed.

In embodiments, at least one of the first heterologous polypeptide coding sequences or the second heterologous polypeptide coding sequence replaced an entire host nematode gene ortholog at a native locus. In certain embodiments, each of the first and second heterologous polypeptide coding sequences individually replaced an entire host nematode gene ortholog at a native locus. In certain embodiments, the host nematode ortholog gene of the first heterologous polypeptide coding sequence and/or the second heterologous polypeptide coding sequence has been knocked-out.

In embodiments, the first and second heterologous polypeptide coding sequences comprise human exon coding sequences. In certain embodiments, the human genes are selected from those listed in Table 1, Table 3 or Example 3. In embodiments, the chimeric heterologous polypeptide coding sequence is integrated in the nematode genome. In certain embodiments, the chimeric heterologous polypeptide coding sequence is inserted into a native locus of the host nematode. In alternative embodiments, the chimeric heterologous polypeptide coding sequence is inserted into a non-native locus of the host nematode or is inserted into a random site of the host nematode genome.

In embodiments, at least one of the first heterologous polypeptide coding sequence or the second heterologous polypeptide coding sequence comprise one or more mutations in the heterologous polypeptide coding sequence exon coding sequences as compared to a wildtype reference sequence resulting in at least one amino acid change when the first heterologous polypeptide coding sequence or the second heterologous polypeptide coding sequence is expressed. In embodiments, the mutation corresponds to a human disease gene clinical variant.

In embodiments, the heterologous phenotype is a monogenic human disease phenotype. In certain other embodiments, the heterologous phenotype is a polygenic human disease phenotype. In embodiments, the heterologous polypeptide coding sequence is a human gene, and in certain embodiments, the heterologous polypeptide coding sequence is a human disease gene.

In embodiments provided herein is a transgenic nematode system for assessing a heterologous disease phenotype, wherein the system comprises a host nematode comprising and expressing a first heterologous polypeptide coding sequence and a second heterologous polypeptide coding sequence, wherein the first and second heterologous polypeptide coding sequence(s) are integrated into the host nematode genome, wherein at least one of the first heterologous polypeptide coding sequence or the second heterologous polypeptide coding sequence comprises one or more mutations in the heterologous exon coding sequence as compared to a wildtype reference sequence resulting in at least one amino acid change when the first heterologous polypeptide coding sequence or the second heterologous polypeptide coding sequence is expressed, and wherein expression of the first and second heterologous polypeptide coding sequences contribute to the heterologous disease phenotype.

In certain embodiments provided herein is a humanized transgenic nematode system for assessing a monogenic or polygenic human disease phenotype, wherein the system comprises a host nematode comprising and expressing a first human polypeptide coding sequence and a second human polypeptide coding sequence, wherein the first and second human polypeptide coding sequences are integrated into the host nematode genome, wherein at least one of the first human polypeptide coding sequence or the second human polypeptide coding sequence comprises one or more mutations in the human gene exon coding sequence as compared to a wildtype reference sequence resulting in at least one amino acid change when the first human polypeptide coding sequence or the second human polypeptide coding sequence is expressed, and wherein expression of the first and second human polypeptide coding sequences contribute to the monogenic or polygenic human disease phenotype.

In embodiments, at least one, or each, heterologous polypeptide coding sequences (e.g., first, second, or additional heterologous polypeptide coding sequence) is present as a single copy providing a heterozygote transgenic nematode. In certain embodiments, the heterozygote is maintained by labeling each chromosome with a marker.

In embodiments, the transgenic nematode systems are used to assess function of the heterologous phenotype resulting from expression of the first and second heterologous polypeptide coding sequence. Those polypeptide coding sequences may be a wildtype sequence (e.g. human sequence) or a clinical variant thereof, wherein the system may be used as a screen for therapeutic agents to identify drugs that may be used to treat individuals with those heterologous phenotype and/or clinical variants. In certain embodiments, the method comprises culturing a host transgenic nematode wherein at least one of the first and second heterologous polypeptide coding sequence is a human clinical variant; and, performing a phenotypic screen to identify a monogenic or polygenic phenotype of the transgenic nematode, wherein a change in phenotype as compared to a control transgenic animal (validated transgenic animal) comprising a corresponding wildtype human heterologous polypeptide coding sequence(s) indicates an altered function of the clinical variant in the transgenic host nematode.

In embodiments, the phenotypic screen is selected from a measurement of electrophysiology of pharynx pumping, a food race, lifespan extension and contraction assay, movement assay, fecundity assay with egg lay or population expansion, apoptotic body formation, chemotaxis, lipid metabolism assay, body morphology changes, fluorescence changes, drug sensitivity and resistance assays, oxidative stress assay, Endoplasmic Reticulum stress assay, nuclear stress assay, response to vibration, response to electric shock, or a combination thereof. In certain embodiments, the identified phenotype is selected from electropharyngeogram variant, feeding behavior variant, defecation behavior variant, lifespan variant, electrotaxis variant, chemotaxis variant, thermotaxis variant, mechanosensation variant, movement variant, locomotion variant, pigmentation variant, embryonic development variant, organ system morphology variant, metabolism variant, fertility variant, dauer formation variant, stress response variant, or a combination thereof.

### BRIEF DESCRIPTION OF THE DRAWINGS

FIG. 1 is an illustration of SNARE genes and their associated presynaptic proteins. SNARE proteins act as machinery to cause vesicle fusion (syntaxins, VAMPs and SNAPs). A set of additional proteins regulate vesicle fusion to coordinate neurotransmitter release with membrane depolarization events.

FIG. 2 shows expected electrophysiology of a wildtype control nematode (black bar) and transgenic nematodes comprising humanized synapse genes following replacement of host SNARE genes with human SNARE genes (e.g. STX1A, SNAP25 and VAMP2, individually (hollow box bar) and additive as STX1A, SNAP25 and VAMP2 humanized complex (grey bar)).

FIG. 3 is an illustration of genes involved in homologous recombination (HR). Five events are involved in activation of HR. Recognition recognizes double strand break (DSB) damage and recruits other recognition partners, RBBP8, BARD1, BRCA1 and BRIP1. Resection is an activity to removed DNA from DSBs by the activity of RAD50, MRE11A and NBN. Filament is the formation of a primed end via the activity of RPA with RAD51 paralogs. Strand invasion creates crossovers into sister chromosome by activity of RAD54. Resolution is an activity mediated by POLD1 with contribution from BLM, TOP3A and MUS81 to synthesize new DNA then ligate back to original chromosome.

FIG. 4 shows expected fluorescence signal from homologous-recombination-activity-activated fluorescent reporter. Wildtype control nematode (black bar) and transgenic nematodes comprising humanized HR apparatus genes (e.g. ATM, RAD50, RAD51, RAD54, and POLD1 individually (hollow box bar) and additive as ATM, RAD50, RAD51, RAD54 and POLD1 humanized complex (grey bar)).

### DETAILED DESCRIPTION OF THE INVENTION

### Introduction

Provided herein is a transgenic non-human animal system, and uses thereof for assessing a heterologous phenotype (polygenic or monogenic) wherein a host animal of the system comprises (and expresses) a plurality (e.g. at least a first heterologous polypeptide coding sequence and a second heterologous polypeptide coding sequence) heterologous polypeptide coding sequences, wherein expression of those polypeptide coding sequences contribute to the heterologous phenotype in the host animal due to their interrelated function as they relate to an observable phenotype. In embodiments, the non-human transgenic animal is a nematode or zebrafish. The present transgenic non-human animal system provides a model for assessing both monogenic and polygenic diseases, wherein a plurality of interrelated heterologous polypeptide coding sequences are expressed and, interact in vivo to provide an observable phenotype. In embodiments, each of the at least two heterologous polypeptide coding sequences comprise wild type coding sequences, for example a common allele of a human gene. In certain other embodiments, at least one of the heterologous polypeptide coding sequences (e.g. a first heterologous polypeptide coding sequences and a second heterologous polypeptide coding sequences) comprise wildtype coding sequence and the remaining heterologous polypeptide coding sequences comprise a variant of a wildtype coding sequence resulting in at least one amino acid change. In certain embodiments, the plurality of heterologous polypeptide coding sequences comprise variant coding sequences. In embodiments, those heterologous polypeptide coding sequences comprise clinal variant coding sequences.

In embodiments, the plurality of heterologous polypeptide coding sequences in the host nematode are integrated into the host genome. In certain embodiments, one or more of the plurality of heterologous polypeptide coding sequences are integrated at a native locus and replace the nematode ortholog. Host nematodes are validated when the heterologous polypeptide coding sequences rescues (or at least partially restores) function of the removed nematode ortholog. As used herein, this method of replacing the host nematode ortholog(s) with the heterologous polypeptide coding sequence(s), may also be referenced as “gene-swap”. U.S. Ser. No. 16/281,988, incorporated in its entirety by reference, discloses a method of optimizing a heterologous polypeptide coding sequences for insertion and expression in a nematode wherein host intron sequences from a highly expressed gene are interspersed into the heterologous exon sequences, codons are optimized for expression in the nematode and any aberrant donor or acceptor sites, which may have been introduced via intron and exon splicing, are removed. That method is one way in which the present transgenic nematodes are made. In embodiments, heterologous polypeptide coding sequences are introduced in sequence until a host nematode comprising a particular number of heterologous polypeptide coding sequences is made. In other embodiments, two or more heterologous polypeptide coding sequences may be introduced into the host nematode genome simultaneously. In other embodiments, transgenic nematodes, each comprising and expressing a single heterologous polypeptide coding sequences are crossed producing progeny with a desired number of unique heterologous polypeptide coding sequences integrated into the host nematode genome. See Example 4.

As used herein, “chimeric heterologous polypeptide coding sequence” refers to a sequence comprising heterologous (to the host animal) exon coding sequences interspersed, or paired, with artificial (or modified) host animal intron sequences, wherein the chimeric heterologous polypeptide coding sequences is optimized for expression in the host animal (e.g. nematode) which may include codon optimization and removal of any aberrant splice donor and/or acceptor sites that were introduced as a function of the chimeric sequences. In embodiments, the heterologous exon coding sequences are “wild type” or from an allele that is reflective of a heterogenous population or a common allele in a population. In certain embodiments, the heterologous exon coding sequences are from human genes. A “validated” transgenic animal system are those animals that have a phenotypic profile that is deemed to have demonstrated rescue or partial restoration of function of the swapped genes, as compared to a control host animal (e.g., wild type (N2) animal that is genetically identical to the host animal prior to the introduction of the heterologous polypeptide coding sequences).

In embodiments, the validated transgenic animal system may be used for assessing the interrelated function of the expressed plurality of heterologous polypeptide coding sequences in host organism.

Provided further is a transgenic animal system for assessing function of one or more variant heterologous polypeptide coding sequences, wherein clinical variants (expressed heterologous polypeptide coding sequences comprising one or more amino acid changes as compared to the wild type heterologous gene) are installed in the heterologous polypeptide coding sequences via site directed mutagenesis. In this instance, the host nematode may comprise two or more heterologous polypeptide coding sequences that comprise clinical variant coding sequences, or the host nematode may comprise one or more heterologous polypeptide coding sequences that comprise wildtype coding sequences and one or more heterologous polypeptide coding sequences that comprise clinical variant coding sequences. Clinical variants are typically classified as pathogenic, likely pathogenic, benign, likely benign or a variant of unknown significance (VUS). The system provides a platform that can be used to test the function of those heterologous polypeptide coding sequences (e.g. human genes), variants of those heterologous polypeptide coding sequences (e.g. human clinical variants), or as a drug screening platform identifying therapeutic agents or drugs that alter the function of the expressed heterologous polypeptide coding sequences or for treatment of animals, including humans (e.g. drug candidates specific to the clinical variants of the heterologous polypeptide coding sequences) in the context of their interaction with other expressed interrelated heterologous polypeptide coding sequences in vivo.

The animals of the invention are “genetically modified” or “transgenic” at multiple loci, which means that they have at least two transgenes, or other foreign DNAs, added or incorporated, or an endogenous gene modified, including, targeted, recombined, interrupted, deleted, disrupted, replaced, suppressed, enhanced, or otherwise altered, to mediate a genotypic or phenotypic effect in at least one cell of the animal and typically into at least one germ line cell of the animal. In some embodiments, the animal may have each of the plurality of transgenes integrated on one allele of its genome (heterozygous transgenic). In other embodiments, animal may have each of the plurality of transgenes on two alleles (homozygous transgenic).

In certain embodiments, the transgenic animals are model organisms including, but not limited to, nematodes, zebrafish, fruit fly, xenopus, or rodents, such as mice and rats.

In certain embodiments, the present transgenic animals provide a plurality of heterologous polypeptide coding sequences, wherein each is a single gene copy wherein a chimeric optimized cDNA of a heterologous polypeptide coding sequence, e.g. modified human cDNA, is inserted to replace coding sequences of a C. elegans ortholog. The humanized nematode is then compared to a nematode lacking the orthologous C. elegans genes, to confirm significant restoration of wild type function. The validated transgenic animal is then modified by installation of at least one clinical variant and tested in one or more phenotyping assays to detect aberrant function. These transgenic animal models have distinct advantages for testing and exploring variant biology. For example, humanized models circumvent differences in compound binding between humans and other species.

In embodiments, the chimeric heterologous polypeptide coding sequences each comprise human heterologous exon coding sequences interspersed, or paired, with artificial host nematode intron sequences optimized for expression in the host nematode. In embodiments, the host nematode intron coding sequences are from a highly expressed C. elegans gene and may be further modified for optimized expression. Provided herein are transgenic nematodes comprising and expressing heterologous polypeptide coding sequences, wherein the host nematode comprises a plurality of chimeric heterologous polypeptide coding sequences comprising heterologous exon coding sequences interspersed with artificial host nematode intron sequences optimized for expression in the host nematode and selected from SEQ ID NO: 1 to 6. In embodiments, the heterologous exon coding sequences are human selected from the human genes of Table 1, Table 3 or Example 3.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized. “Polypeptide coding sequence” as used herein means the nucleic acid coding sequence that encodes for a specific amino acid sequence, such as a heterologous polypeptide.

“cDNA” as used herein means the deoxyribonucleic acid sequence that is derived as a copy of a mature messenger RNA sequence and represents the entire coding sequence needed for creation of a fully functional protein sequence.

As used herein, the terms “disrupt,” “disrupted,” and/or “disrupting” in reference to a gene mean that the gene is degraded sufficiently such that it is no longer functional. In embodiments, the native ortholog gene is replaced with the (chimeric) heterologous polypeptide coding sequence effectively disrupting the native host gene.

“Donor DNA”, “donor template” and “repair template” as used interchangeably herein refers to a double, or single-stranded DNA fragment or molecule that includes at least a portion of the gene of interest. The donor DNA may encode a full-functional protein or a partially-functional protein.

As used herein, the term “donor homology” refers to a sequence at a target edit site that is also include in the nucleic acid sequence of a plasmid DNA construct that is necessary to instruct endogenous homologous repair machinery of the cell to create in frame insertion of a transgene sequence. Typically, a plasmid for instructing transgenesis contains a both a left-side and right-side donor homology sequence

As used herein, the term “gene editing” refers to a type of genetic engineering in which DNA is inserted, replaced, or removed from a genome using gene editing tools. Examples of gene editing tools include, without limitation, zinc finger nucleases, TALEN and CRISPR.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but is not limited to epilepsy, DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease. “Clinical variants” are used herein, are those genes that lead to a genetic disease wherein expression of the gene results in one or more amino acid changes as compared to benign allele that does not lead to disease.

A “heterologous gene” or “heterologous polypeptide coding sequence” as used herein refers to a nucleotide sequence not naturally associated with a host animal into which it is introduced, including for example, exon coding sequences from a human gene introduced, as a (chimeric) heterologous polypeptide coding sequence, into a host nematode. In embodiments, the heterologous polypeptide coding sequence may comprise one or more point mutation(s) which results in one or more amino acid changes in the expressed product, wherein any change as compared to a host wild type sequence is considered a “heterologous polypeptide coding sequence” regardless if the entire sequence, or just one nucleic acid change, was introduced into the host genome.

The term “heterologous polygenic or monogenic phenotype” as used herein, refers to any measurable phenotype that is different as compared to a host “wild-type” phenotype. “Polygenic” and “monogenic” refer to a phenotype that is induced by one (“monogenic”), or more expressed transgenes.

The term “human disease phenotype” as used herein, including both “monogenic” and “polygenic”, refers to an observable phenotype induced by expression of one or more human disease transgenes. In other words, an observable phenotype seen in the host animal after insertion into the genome of a sequence the encodes for a human disease gene, such as a clinical variant. The phenotype may not be related to a phenotype seen in a human with a corresponding genetic disease, but is any observable phenotype that is different, and or distinct, from an observable phenotype of a wild type host animal. The observable human disease phenotype, in the instant disclosure, is used as a readout to enable study of human genetic diseases via a host animal (e.g. nematodes or zebrafish) expressing the disease gene product.

The term “homolog” refers to any gene that is related to a reference gene by descent from a common ancestral DNA sequence. The term “ortholog” refers to homologs in different species that evolved from a common ancestral gene by speciation. Typically, orthologs retain the same or similar function despite differences in their primary structure (mutations).

As used herein, the term “homology driven recombination” or “homology direct repair” or “HDR” is used to refer to a homologous recombination event that is initiated by the presence of double strand breaks (DSBs) in DNA (Liang et al. 1998); and the specificity of HDR can be controlled when combined with any genome editing technique known to create highly efficient and targeted double strand breaks and allows for precise editing of the genome of the targeted cell; e.g. the CRISPR/Cas9 system (Findlay et al. 2014; Mali et al. February 2014; and Ran et al. 2013).

As used herein, the term “enhanced homology driven insertion or knock-in” is described as the insertion of a DNA construct, more specifically a large DNA fragment or construct flanked with homology arms or segments of DNA homologous to the double strand breaks, utilizing homology driven recombination combined with any genome editing technique known to create highly efficient and targeted double strand breaks and allows for precise editing of the genome of the targeted cell; e.g. the CRISPR/Cas9 system. (Mali et al. February 2013).

As used herein, the terms “increase,” “increased,” “increasing,” “improved,” (and grammatical variations thereof), describe, for example, an increase of at least about 5%, 10%, 15%, 20%, 25%, 35%, 50%, 75%, 80%, 85%, 90%, 95%, 97%), 98%), 99%), or 100% as compared to a control. In embodiments, the increase in the context of a heterogenous gene or clinical variant thereof, is measured and/or determined via phenotypic assay to assess function of the expressed gene.

As used herein, the term “genomic locus” or “locus” (plural loci) is the specific location of a gene or DNA sequence on a chromosome and, can include both intron or exon sequences of a particular gene. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, introns, exons, promoter sequences, terminators, translational regulatory sequences such as ribosome binding sites and internal ribosome entry sites, enhancers, silencers, insulators, boundary elements, 5′ or 3′ regulatory sequences, replication origins, matrix attachment sites and locus control regions. As used herein “native locus” refers to the specific location of a host gene (e.g., ortholog to the heterologous polypeptide coding sequence) in a host animal.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. As used herein, “clinical variant” is a disease gene that comprises one or more amino acid changes as compared to wild type and is thus a mutant gene.

A “normal” or “wild type” nucleic acid, nucleotide sequence, polypeptide or amino acid sequence refers to a naturally occurring or endogenous nucleic acid, nucleotide sequence, polypeptide or amino acid sequence that has not undergone a change. As used herein, the wild type sequence may be a disease gene, but does not comprise a mutation leading to a pathogenic phenotype. It is understood there is a distinction between a wild type disease gene (e.g. those without a mutation leading to a pathogenic phenotype and may be an allele reflective of a “normal” heterogenous population) and clinical variants that comprise one or more mutations of those disease genes and that may have a pathogenic phenotype. In embodiments, the normal gene or wild type gene may be the most prevalent allele of the gene in a heterogenous population. N2 are wild type C. elegans nematodes.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein. In embodiments, function is determined via one or more phenotypic assays wherein a phenotypic profile for the mutant (disease) gene may be generated.

As used herein, the term “percent sequence identity” or “percent identity” refers to the percentage of identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence

As used herein, the term “percent sequence similarity” or “percent similarity” refers to the percentage of near-identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent similarity” can refer to the percentage of near-identical amino acids in an amino acid sequence. Near-identical amino acids are residues with similar biophysical properties (e.g., the hydrophobic leucine and isoleucine, or the negatively-charged aspartic acid and glutamic acid).

As used herein, the term “polynucleotide” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA as DNA construct, mRNA, and anti-sense RNA, any of which can be single stranded or double stranded. The terms “polynucleotide,” “nucleotide sequence” “nucleic acid,” “nucleic acid molecule,” and “oligonucleotide” are also used interchangeably herein to refer to a heteropolymer of nucleotides. Except as otherwise indicated, nucleic acid molecules and/or polynucleotides provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%), 98%), 99%), or 100% as compared to a control. In embodiments, the reduction in the context of a heterogenous gene or clinical variant thereof, is measured and/or determined via phenotypic assay to assess function of the expressed gene.

The term “safe harbor” locus as used herein refers to a site in the genome where transgenic DNA (e.g., a construct) can be added whose expression is insulated from neighboring transcriptional elements such that the transgene expression is fully depend on only the introduced transgene regulatory elements. In certain embodiments, the present invention involves incorporation and expression of transgenic DNA includes transgenes within a safe harbor locus.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” and grammatical variations thereof in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%> nucleotide or amino acid residue identity, when compared and aligned for maximum correspondence, as measured using one of the following sequence comparison algorithms or by visual inspection. In particular embodiments, substantial identity can refer to two or more sequences or subsequences that have at least about 70%, at least about 75%, at least about 80%, at least about 85%, at least about 90%, at least about 95, 96, 96, 97, 98, or 99% identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Subject” and “patient” as used herein interchangeably refers to any vertebrate, including, but is not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. The subject or patient may be undergoing other forms of treatment. In embodiments, the patient is a human wherein a clinical variant is a sequence of a disease gene from the patient.

“Target gene” as used herein refers to any nucleotide sequence encoding a known or putative gene product. As used herein the target gene may be the (chimeric) heterologous polypeptide coding sequence, either in normal or wild type form, or as a clinical variant, or the host animal ortholog of the heterologous polypeptide coding sequence. The target gene may be a mutated gene involved in a genetic disease, also referred to herein as a clinical variant.

“Target nucleotide sequence” as used herein refers to the region of the target gene to which the Type I CRISPR/Cas system is designed to bind.

The terms “transformation,” “transfection,” and “transduction” as used interchangeably herein refer to the introduction of a heterologous nucleic acid into a cell. Such introduction into a cell may be stable or transient. Thus, in some embodiments, a host cell or host organism is stably transformed with a polynucleotide of the invention. In other embodiments, a host cell or host organism is transiently transformed with a polynucleotide of the invention. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromasomally, for example, as a mini-chromosome or a plasmid. In certain embodiments, the nucleotide sequences, constructs, expression cassettes can be expressed transiently and/or they can be stably incorporated into the genome of the host organism, such as in a native, non-native locus or safe harbor location.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

The term “3′untranslated region” or“3′UTR” refers to a nucleotide sequence downstream (i.e., 3′) of a coding sequence. It generally extends from the first nucleotide after the stop codon of a coding sequence to just before the poly(A) tail of the corresponding transcribed mRNA. The 3′ UTR may contain sequences that regulate translation efficiency, mRNA stability, mRNA targeting and/or polyadenylation. In embodiments, the 3′ UTR may be native, or non-native in the context of the (chimeric) heterologous polypeptide coding sequence.

“Variant” with respect to a peptide or polypeptide that differs in one or more amino acid sequence by the insertion, deletion, or conservative substitution of amino acids as compared to a normal or wild type sequence. The variant may further exhibit a phenotype that is quantitatively distinguished from a phenotype of the normal or wild type expressed gene. In embodiments, clinical variant refers to a disease gene with one or more amino acid changes as compared to the normal or wild type disease gene.

Transgenic Nematodes

The instant transgenic nematode system comprises a host nematode that comprises and expresses a first heterologous polypeptide coding sequence and a second heterologous polypeptide coding sequence. As used herein, at least a first heterologous polypeptide coding sequence and second heterologous polypeptide coding sequence, may be referred to collectively as a “plurality” of heterologous polypeptide coding sequences. The present transgenic nematodes comprise at least two distinct heterologous polypeptide coding sequences that are interrelated as to an observable phenotype, such as a monogenic or polygenic disease. As used herein “distinct heterologous polypeptide coding sequences” means a sequence that codes for a unique protein wherein each are under control of a separate promotor and/or other regulatory elements. In embodiments, the plurality of heterologous polypeptide coding sequences do not include a reporter gene or a prokaryotic gene. In embodiments, the first and second heterologous polypeptide coding sequences are integrated into the host nematode genome, wherein expression of the first and second heterologous polypeptide coding sequences contribute to the heterologous phenotype.

The present host nematodes comprise at least two (“digenic”) heterologous polypeptide coding sequences, wherein their expression products, directly or indirectly, are interrelated such as in a pathway (e.g. homologous recombination) or a disease phenotype (e.g. autism, epilepsy or neurodegenerative disorder). In many instances a variant of pathogenic consequence occurs at a protein-protein interaction domain, therefore modeling a pathogenic variant in a single gene humanized animal will be insufficient for creating a condition in which pathogenic behavior can be detected. At a minimum, at least two human genes need to be installed in the host animal genome so the protein-protein interaction variant can be modeled in vivo. In other conditions, the pathogenic behavior will only manifest if two genes in a pathway are humanized so that polygenic additive effects reaching a pathogenicity threshold can be observed. As a result, multiple polypeptide coding sequences need to be installed so that proper protein complex, pathway signaling, and/or metabolic processes can be faithful recapitulates as observed in the human condition.

In embodiments, the host nematode comprises and expresses additional heterologous polypeptide coding sequences that are also interrelated as to the first and second heterologous polypeptide coding sequences. In embodiments, the present host nematodes comprise and express from two (2) to about fifteen (15) heterologous polypeptide coding sequences, optionally from three (3) to about fifteen (15) polypeptide coding sequences. Those plurality of heterologous polypeptide coding sequences may individually code for a wild type sequence or a variant thereof including identified clinical variants. It is also an aspect of the invention that the host transgenic nematodes, in addition to the plurality of heterologous polypeptide coding sequences, comprise and/or express a reporter heterologous polypeptide coding sequences.

In embodiments, one or more of the plurality of heterologous polypeptide coding sequences is a (chimeric) heterologous polypeptide coding sequence. As used herein “chimeric heterologous polypeptide coding sequence” refers to a sequence comprising heterologous exon coding sequences and host animal (e.g. nematode) intron sequences interspersed or paired with the exon coding sequences. In embodiments, the heterologous polypeptide coding sequence corresponds to a nematode ortholog, wherein the chimeric heterologous polypeptide coding sequence replaced the entire host nematode ortholog, either prior to or at the same time the chimeric heterologous polypeptide coding sequence is installed, and wherein the chimeric heterologous polypeptide coding sequence is installed at the host nematode ortholog native locus. In embodiments, each of the heterologous polypeptide coding sequences are integrated into the native locus of the nematode as a chimeric heterologous polypeptide coding sequence. It is not an aspect of the invention for partial removal with partial replacement, of the host animal ortholog. Further, the plurality of interrelated heterologous polypeptide coding sequences are eukaryotic; it is not an aspect of the invention for the plurality of interrelated heterologous polypeptide coding sequences to be prokaryotic. In embodiments, the host nematode is a C. elegans, C. briggsae, C remanei, C. tropicalis, or P. pacificus. (Sugi T et al. Genome Editing in C. elegans and Other Nematode Species. Int J Mol Sci. 2016 Feb 26; 17(3):295).

In embodiments, the plurality of heterologous polypeptide coding sequences are selected from a different species of nematode (e.g. parasitic nematode), an avian, mammal or fish. In certain embodiments, the plurality of heterologous polypeptide coding sequences are human. In embodiments, the heterologous polypeptide coding sequences replace the entire nematode ortholog gene at their respective native loci, accordingly the heterologous polypeptide coding sequences must have a homolog as an identified ortholog in the host nematode. In one embodiment, the homolog is of substantial quality when sequence identity between heterologous source and host exceeds 70%. In one embodiment, the homolog is of high quality when sequence identity between heterologous source and host exceeds 50%. In other embodiments, the homolog is good when its identity exceeds 35%. In other embodiments, the homolog is adequate when its identity exceeds 20%. In other embodiments, the homolog is poor but acceptable when its identity is less than 20%. See Example 1 for identification of host nematode orthologs; and, Tables 1 and 3 for a pairing of human polypeptide coding sequences and nematode orthologs.

In alternative embodiments, the plurality of heterologous polypeptide coding sequences are from a parasitic nematode, which are selected from Trichuris muris, Ascaris lumbricoides, Ancylostoma duodenale, Necator americanus, Trichuris trichiura, Enterobius vermicularis, Strongyloides stercoralis, Trichinella spiralis, Wuchereria bancrofti, Brugia malayi, Brugia timori, Loa loa, Mansonella streptocerca, Onchocerca volvulus, Mansonella perstans, Mansonella ozzardi, Cooperia punctata, Cooperia oncophora, Ostertagia ostertagi, Haemonchus contortus, Ascaris suum, Aphelenchoides, Ditylenchus, Globodera, Heterodera, Longidorus, Meloidogyne, Nacobbus, Pratylenchus, Trichodorus, Xiphinema, Bursaphelenchus, Dirofilaria immitis, Toxocara canis, Toxocara cati, Ancylostoma braziliense, Ancylostoma tubaeforme, Ancylostoma caninum, Dirofilaria repens, and Uncinaria stenocephala.

In certain embodiments, the plurality of heterologous polypeptide coding sequences are human polypeptide coding sequences. In certain embodiments, the human polypeptide coding sequences are wild type polypeptide coding sequences. Provided herein is a transgenic nematode system comprising a host nematode comprising a plurality of chimeric heterologous polypeptide coding sequences optimized for expression in the host nematode wherein the heterologous polypeptide coding sequences replace their respective host nematode gene ortholog and the heterologous polypeptide coding sequences rescues, or at least partially restores, function of the replaced nematode orthologs. Heterologous polypeptide coding sequences that rescue function of the replaced nematode ortholog are referred to herein as “wild type” heterologous polypeptide coding sequences.

In other embodiments, the plurality of heterologous polypeptide coding sequences are human disease genes. As used herein, “disease gene” or “disease polypeptide coding sequence” refers to a gene or expressed sequence involved in or implicated in a disease. In certain embodiments provided herein are transgenic nematodes comprising a plurality of heterologous polypeptide coding sequences that are human wild type disease genes that have replaced the host nematode orthologs at their native loci. See Examples 1 to 4. Those human heterologous disease polypeptide coding sequences represent targets for drug discovery and drugs that rescue function of human clinical variants.

In embodiments, the heterologous polypeptide coding sequences rescue, or at least partially restore, function of the removed host nematode orthologs. Rescue or restoration of function, which is measured in a phenotypic assay, identifies those transgenic nematodes that are validated and may be used as a transgenic control animal. As used herein “validated transgenic control nematode” means a transgenic nematode expressing a plurality of chimeric heterologous polypeptide coding sequences in place of host nematode orthologs, wherein at least partial function is rescued by expression of the heterologous polypeptide coding sequences. Rescued function can be from 1% to 100% as compared to a host nematode expressing the heterologous “wild-type” polypeptide coding sequence. In other embodiments, rescued function can be from 1% to 100% as compared to a host nematode with a knock-out of the ortholog.

In addition to quantitative rescue effects, rescue can be qualitative as to essential genes, wherein rescue with a heterologous transgene provides sufficient lifespan and fecundity for establishment of a propagating colony.

In embodiments, rescue of function is measured by analyzing, observing or monitoring the transgenic nematodes in a phenotypic assay as compared to host nematodes (KO of ortholog sequence or expressing the heterologous wild type polypeptide coding sequence) and/or null variants. In embodiments, the phenotypic assay is selected from a measurement of electrophysiology of pharynx pumping, a food race, lifespan extension and contraction assay, movement assay, fecundity assay with egg lay or population expansion, apoptotic body formation, chemotaxis, lipid metabolism assay, body morphology changes, fluorescence changes, drug sensitivity and resistance assays, or a combination thereof. There is no limitation as to the phenotypic assay that may be used, including those developed in the future, provided a useful phenotype profile can be generated for assessing function of the installed heterologous polypeptide coding sequence. The above are representative phenotype assays, but others may be used to validate the transgenic nematode, as well as for assessing variants of the heterologous polypeptide coding sequences.

In embodiments, a phenotype profile of the transgenic nematode is identified from the assay wherein the identified phenotype is selected from electropharyngeogram variant, feeding behavior variant, defecation behavior variant, lifespan variant, electrotaxis variant, chemotaxis variant, thermotaxis variant, mechanosensation variant, movement variant, locomotion variant, pigmentation variant, embryonic development variant, organ system morphology variant, metabolism variant, fertility variant, dauer formation variant, stress response variant, or a combination thereof.

In certain embodiments provided herein are validated transgenic control nematodes of the present system, comprising a plurality of heterologous polypeptide coding sequences optimized for expression in the host nematode wherein the heterologous polypeptide coding sequences replace their respective host nematode gene orthologs and the heterologous polypeptide coding sequences rescue function of the replaced nematode orthologs. In embodiments, the heterologous polypeptide coding sequences are human disease genes.

In embodiments, the transgenic nematodes further comprise an inducible reporter gene operably linked to an inducible promoter. See U.S. Pat. No. 8,937,213, herein incorporated by reference, which disclose use of inducible and constitutive promoters operably linked to reporter genes. Reporter genes are well known in the art and include luminescent and fluorescent proteins that can be expressed in living cells. Well known examples include GFP, mCherry, mTurquoise and mVenus. In certain embodiments the inducible promoter is from a gene induced by the heterologous polypeptide coding sequence, or the variant heterologous polypeptide coding sequence. In certain embodiments, the inducible promoter is from a gene inhibited by the variant heterologous polypeptide coding sequence.

The present validated transgenic nematodes are prepared via homologous recombination at the native locus of the host nematode ortholog wherein a plurality of nematode orthologs are replaced with the heterologous polypeptide coding sequences. This method is advantageous in that it provides a platform for further testing and modifications and provides an improvement over previously disclosed methods that use amino acid substitution for generation of humanized nematodes expressing clinical variants. The use of gene-swap (i.e. heterologous polypeptide coding sequence replaces the nematode ortholog at the native locus) avoids the expression level issues that are a challenging problem with extrachromosomal array studies. Instead, CRISPR techniques are deployed to directly mutate at native loci (Farboud B and Meyer BJ. Dramatic enhancement of genome editing by CRISPR/Cas9 through improved guide RNA design. Genetics. 2015 April; 199(4):959-71; Paix A et al. High Efficiency, Homology-Directed Genome Editing in Caenorhabditis elegans Using CRISPR-Cas9 Ribonucleoprotein Complexes. Genetics. 2015 September; 201(1):47-54).

Gene swap involves removal of the native coding sequence of the host nematode (e.g. C. elegans) ortholog and replacement with cDNA from the heterologous polypeptide coding sequence (e.g., human gene), wherein the exon coding sequences of the heterologous polypeptide coding sequence are paired with, or interspersed with, host nematode intron sequences. The host intron sequences are derived from a highly expressed host gene and may be further modified for expression of the heterologous exon coding sequences. As used herein “chimeric heterologous polypeptide coding sequence” refers to a sequence of heterologous (to the host animal) exon coding sequences that are paired or interspersed with the host animal intron sequences. Representative modified host nematode intron sequences are selected from SEQ ID NO: 1 to 6. In certain embodiments, the present transgenic nematodes comprise a chimeric heterologous polypeptide coding sequence comprising one or more of SEQ ID NO: 1 to 6. Those sequences, when used with human exon coding sequences have demonstrated good expression in a host nematode.

To execute a gene-swap, the coding sequence from heterologous cDNA is optionally adjusted for optimal expression in the host nematode, e.g., C. elegans. In addition to the use of host animal intron sequences paired with heterologous exon coding sequences, optimization includes codon optimization for the host animal and removal of any aberrant splice donor and/or acceptor sites that were generated as a result of the chimeric sequence. Accordingly, in embodiments provided herein are transgenic nematodes comprising a chimeric heterologous polypeptide coding sequences optimized for expression in the host nematode wherein a heterologous polypeptide coding sequence replaces a host nematode gene ortholog, wherein the chimeric heterologous polypeptide coding sequence comprises heterologous exon coding sequences interspersed with artificial host nematode intron sequences.

In embodiments, optimization comprises codon optimization (e.g. removal of rare codons), introduction of host intron sequences into the heterologous cDNA and removal of any aberrant splice sites. For codon optimization, rare codon usage must be avoided to enable sufficient levels of protein translation from a mRNA message. For intron sequences, the artificial host intron sequences are added to the codon optimized heterologous cDNA sequence, which results in improved mRNA stability, and a chimeric sequence. Performing those techniques are well known in the art and online tools exist for performing both. Conveniently, codon optimization and identification of aberrant splice sites are achieved with the C. elegans codon adapter that encodes optimal amino acid sequence (Redemann S et al., C. elegans codon Adapter— GGA, Nat Methods. 2011 Mar.; 8(3):250-2) and NextGene2 which adjust splice donor and acceptor sites for optimal performance (Hebesgaard S M et al., Nucleic Acids Res. 1996 Sep 1; 24(17):3439-52).

Those chimeric sequences, heterologous cDNA optimized, and artificial host intron sequences added may result in a sequence with highly repetitive sequences that prevent gene synthesis by DNA sequence providers. As a result, the sequence may be hand curated to minimize repeat sequence formation and enable synthesis to proceed from suppliers. The need to hand curate sequence content creates a need for removal of aberrant splice site donor and acceptor site. Online tools exist for identify unintentional splice site donor and acceptor sites (Hebesgaard S M et al., Nucleic Acids Res. 1996 Sep 1; 24(17):3439-52). Additional hand curated sequence adjustments are made iteratively until on-line software no longer detects aberrant splice site donor and acceptor sites. Because a given optimization may fail to express properly for unforeseen reasons, three sets of expression-optimized human cDNA are frequently made so that at least three attempts at null rescue can be attempted.

In embodiments, the intron sequences provided by the C. elegans codon Adapter are synthetic introns that are not ideal for expression. However, the synthetic host intron sequences can be modified to meet certain criteria optimal for expression of the heterologous polypeptide coding sequence. Those criteria include intron sequences, for expression in a host nematode such as C. elegans, that are: from a gene highly expressed native C. elegans genes; small (less than 80 bp); do not contain stop codons; are divisible by 3; and, have a low hydropathy index. Host intron sequences that do not meet those criteria can be modified by deleting or changing bases. Host intron sequences meeting the above criteria are likely to not negatively affect gene expression or plasmid building and at the same time, even if un-spliced in synthetic DNA, will retain reading frame and code for peptides with low hydrophobicity content. As a result, functional protein is likely even if all the intron sequences fail to splice.

In some embodiments, the intron position is based on the protein structure. Protein structure can be identified by using published data such as X-ray crystallography. An alignment of orthologs and paralogs is performed. Un-conserved regions are mapped to the structure to find loop regions. The target gene is labeled for loop regions. Amino acid pairs are identified in the loop region that can be coded for a good splice donor and acceptor such as KE, KD, QE, QD, EE, ED, KV, QV, and EV. The introns as disclosed above are inserted between the splice donor and acceptor and the sequence is checked for aberrant splicing as disclosed above.

In certain embodiments, the transgenic control nematodes may be prepared by methods other than homologous recombination into the native locus of the nematode, provided the cDNA of the plurality of heterologous polypeptide coding sequences are optimized for expression in the host nematode by codon optimization, addition of host intron sequences to the cDNA sequence of the heterologous polypeptide coding sequence and removing aberrant splice donor and acceptor sites. Those alternative methods comprise inserting the optimized chimeric heterologous polypeptide coding sequences via homologous recombination into a native locus of the nematode wherein a nematode gene orthologs are removed, wherein the heterologous polypeptide coding sequences are rescued, or at least partially restored, for function of the removed nematode orthologs; or, inserting the optimized heterologous polypeptide coding sequences into a non-native locus of the nematode; or, inserting the optimized heterologous polypeptide coding sequences into a random site of the nematode genome; or, adding the optimized heterologous polypeptide coding sequences as an expression vector wherein the optimized heterologous polypeptide coding sequences are not integrated into the nematode genome.

In embodiments are provided transgenic test nematodes, which are based on the validated transgenic control nematode and comprise a variant of a heterologous polypeptide coding sequence. As used herein, “variant heterologous polypeptide coding sequences” refers to an expressed gene with one or more amino acid changes as compared to a heterologous polypeptide coding sequence that was used to prepare the validated transgenic control nematode. Accordingly, a transgenic test nematode comprises a transgenic control nematode that is a modified validated transgenic nematode, wherein an expressed heterologous polypeptide coding sequence comprises one or more amino acid changes providing a variant of the heterologous polypeptide coding sequence. The transgenic test nematodes may be used for assessing function of the heterologous variant polypeptide coding sequence and drug discovery. In embodiments, a transgenic test nematode comprises a purality of (chimeric) variant heterologous polypeptide coding sequences, comprising heterologous exon coding sequences interspersed with artificial host nematode intron sequences optimized for expression in the host nematode, wherein the exon coding sequences comprise one or more mutations resulting in an amino acid change as compared to a wildtype reference sequence (wild type heterologous polypeptide coding sequence of transgenic control animal), and wherein the (chimeric) variant heterologous polypeptide coding sequence replaces the entire host nematode gene ortholog at a native locus, and wherein the heterologous polypeptide coding sequences is a eukaryotic gene.

In embodiments, a variant heterologous polypeptide coding sequence may be introduced by amino acid swap of the transgenic control nematode or by gene swap of a variant containing heterologous polypeptide coding sequence in as replacement of the native coding sequence. In embodiments, the variant heterologous polypeptide coding sequences is a human disease gene comprising one or more amino acid changes as compared to the wild type disease gene. In embodiments, the variant comprises a single amino acid change wherein the change was installed into the integrated heterologous polypeptide coding sequence of the transgenic control animal via a co-CRIPSR method. The resulting transgenic animals are transgenic test animals (e.g. nematode or zebrafish). In certain embodiments, the mutations (of the heterologous exon coding sequence) are created from a pool of DNA repair templates each containing one or more mutations. In other embodiments, the variant comprises more than one amino acid change. In certain embodiments, those mutations are created from a pool of DNA repair templates each containing two or more mutations. Variants with more than one amino acid change, as compared to the wild type gene, may be a known clinical variant or a combination of two or more variants of the same gene. The combination of clinical variants in one variant heterologous transgenic test animal may be beneficial for assessing function of variants as to their synergistic, antagonistic, additive etc. function as measured in phenotypic assays.

Like drosophila studies, electrophysiology measurements in C. elegans on functional variants can provide a rich and diverse set of phenotyping data (Sorkaç A et al. In Vivo Modelling of ATP1A3 G316S-Induced Ataxia in C. elegans Using CRISPR/Cas9-Mediated Homologous Recombination Reveals Dominant Loss of Function Defects. PLoS One. 2016 Dec 9; 11 (12)). These published studies were done by making “humanizing” mutations at native loci. A homology alignment is used to determine where conserved positions occur between the human gene and its animal model ortholog. Clinical variants are then mapped to the sequence alignment and, if they occur at a conserved amino acid, the clinical variant can be installed by CRISPR as an amino-acid-swap which substitutes the native amino acid with the amino acid change seen in the patient.

In embodiments, the variant heterologous polypeptide coding sequences are human clinical variants. Accordingly, when at least partial rescue of function is achieved with expression of a plurality of heterologous polypeptide coding sequences, the system (comprising validated transgenic nematodes) becomes valid for installation of clinical variants (test transgenic nematodes). Six classes of clinical variants can be installed (Pathogenic, Likely Pathogenic, Uncertain Significance, Likely Benign, Benign, and the unassessed). On average, dbSNP data indicates 80% of known variants are unassessed and nearly half (40%) of the remaining assessed variants are Variants of Uncertain Significance (VUS). (NCBI) Variation Viewer. Installation of known Pathogenic and Benign variants helps determine how conserved are the existing assignments when installed into the human cDNA expressing nematode model. When most of the pathogenic and benign variants give expected activities (e.g., phenotype) in the humanize nematode model the system then is valid for assessment of pathogenicity of VUS and unassigned variants.

In embodiments, methods are provided herein for assessing function of a human clinical variant, comprising the steps of culturing a test transgenic nematode, wherein at least one of variant heterologous polypeptide coding sequences contains human clinical variant; and, performing a phenotypic screen to identify a phenotype of the test transgenic nematode, wherein a change in phenotype as compared to a control transgenic nematode comprising of wildtype heterologous polypeptide coding sequences (e.g. corresponding validated transgenic nematode) indicates an altered function of the clinical variant in the test transgenic nematode. The phenotypic screens and identified phenotypes are disclosed above and are the same as those used when validating the transgenic control nematode for rescue of function.

In embodiments, the phenotypic screen is a food race wherein decreased time to reach food, as compared to the control transgenic nematode, indicates pathogenicity of the human clinical variant. In embodiments, the methods further comprise classifying the human clinical variant as pathogenic, likely pathogenic, uncertain significance, likely benign, or benign following the phenotypic screen.

In certain embodiments, the transgenic test nematode comprises an inducible promoter operably linked to a reporter gene, wherein the promoter is from a gene induced by expression of the human clinical variant gene, wherein the method for assessing function of a human clinical variant comprises culturing a test transgenic nematode, wherein the variant heterologous polypeptide coding sequence is a human clinical variant and, observing the inducible report gene expression, whereby human clinical variant genes with altered function are identified as pathogenic or likely pathogenic when the inducible reporter gene is expressed.

In further embodiments provided herein are methods using the transgenic test nematode system for drug screening. For humanized platforms exhibiting pathogenic activity with a given installed variant, screens of novel and existing compounds can be performed in efforts to find drug candidates with capacity to restore function back towards wild type. In embodiments, the methods for screening therapeutic agents to treat altered function of a human clinical variant, comprises placing a test transgenic nematode in a medium comprising a test compound, wherein a variant heterologous polypeptide coding sequence is a human clinical variant identified as pathogenic, likely pathogenic, unknown significance or unassigned; incubating the test transgenic nematode with the test compound for a period from 2 minutes to 7 hours, or from 1 to 7 days including 1 day, 2 days, 3 days, 4 days, 5 days, 6 days or 7 days; and, performing a screening assay, whereby therapeutic agents are identified from the test compounds when the outcome of the screening assay is deemed positive. An altered phenotype back towards wildtype is considered positive. The screening assays are phenotypic assays disclosed above, including fluorescent assay wherein transgenic test nematode further comprises an inducible promoter operably linked to a reporter gene wherein the promoter is from a gene inhibited in response to expression of the human clinical variant, whereby therapeutic agents are identified when the inducible reporter gene is expressed.

In embodiments provided herein are methods for screening therapeutic agents to treat altered function of a human clinical variant. Those methods comprise use of a present transgenic test animal. In certain embodiments, those methods comprise placing a present transgenic test nematode, with an identified behavioral or molecular phenotype that is different from an identified phenotype of a control transgenic nematode expressing a wildtype heterologous polypeptide coding sequence, in a medium comprising a test compound, wherein the variant heterologous polypeptide coding sequence is a human clinical variant; incubating the test transgenic nematode with the test compound for a period from 2 minutes to seven days, including 1 day, 2 days, 3 days, 4 days, 5 days, 6 days or 7 days; and, performing a phenotypic assay to identify a post-test compound behavioral or molecular phenotype of the test transgenic nematode, whereby therapeutic agents are identified from the test compounds when the post-test compound phenotype is more similar, as compared to the phenotype of the test transgenic nematode, to the phenotype of the control transgenic nematode.

### SPECIFIC EMBODIMENTS

In certain embodiments, provided herein is a non-human animal transgenic system for assessing a heterologous polygenic or monogenic phenotype, comprising: a host non-human animal comprising and expressing a first heterologous polypeptide coding sequence and a second heterologous polypeptide coding sequence, wherein the first and second heterologous coding sequences are integrated into the host animal genome, and wherein expression of the first and second heterologous polypeptide coding sequences in the animal contribute to the heterologous phenotype. In embodiments, the host non-human animal is a nematode or a zebrafish. In certain embodiments, at least one of the first heterologous polypeptide coding sequence or the second heterologous polypeptide coding sequence is a chimeric heterologous polypeptide coding sequence comprising heterologous exon coding sequences interspersed with artificial host intron sequences optimized for expression in the host. In embodiments, each of the first and second heterologous polypeptide coding sequences is individually a chimeric heterologous polypeptide coding sequence comprising heterologous exon coding sequences interspersed with artificial host intron sequences optimized for expression in the host animal. In embodiments, at least one of the first heterologous coding sequence or the second heterologous coding sequence replaced an entire host gene ortholog at a native locus. In embodiments, each of the first and second heterologous coding sequences individually replaced an entire host gene ortholog at a native locus. In embodiments, a host ortholog gene sequence corresponding to the first heterologous coding sequence and/or the second heterologous coding sequence has been knocked-out. In embodiments, the first and second heterologous coding sequences comprise human exon coding sequences. In other embodiments, at least one of the first heterologous polypeptide coding sequence or the second heterologous polypeptide coding sequence comprises one or more mutations in the first and/or second heterologous polypeptide coding sequence coding sequences as compared to a wildtype reference sequence resulting in at least one amino acid change in the first and/or second polypeptide coding sequences when the one or more additional heterologous polypeptide coding sequence is expressed in the host, optionally wherein the mutation corresponds to a human disease gene clinical variant. In some embodiments, the present system further comprises and expresses one or more additional heterologous polypeptide coding sequence that contributes to the heterologous phenotype, optionally wherein the one or more additional heterologous polypeptide coding sequences comprises one or more mutations in polypeptide coding sequence as compared to a wildtype reference sequence resulting in at least one amino acid change when the one or more additional heterologous polypeptide coding sequence is expressed in the host; or optionally wherein the host animal comprises and expresses 3 to 15 heterologous polypeptide coding sequences, wherein optionally a host ortholog gene corresponding to each of the heterologous polypeptide coding sequences has been knocked-out. In certain embodiments, the heterologous phenotype is a monogenic human disease phenotype or alternatively a polygenic human disease phenotype.

In certain embodiments provided herein is a non-human animal transgenic system for assessing a heterologous disease phenotype, comprising: a host animal comprising and expressing a first heterologous polypeptide coding sequence and a second heterologous polypeptide coding sequence, wherein the first and second heterologous polypeptide coding sequences are integrated into the host genome, wherein at least one of the first heterologous polypeptide coding sequence or the second heterologous polypeptide coding sequence comprises one or more mutations in the heterologous polypeptide coding sequence as compared to a wildtype reference sequence resulting in at least one amino acid change when the first heterologous polypeptide coding sequence or the second heterologous polypeptide coding sequence is expressed, and wherein expression of the first and second heterologous polypeptide coding sequence contribute to the heterologous disease phenotype. In embodiments, at least one of the first heterologous polypeptide coding sequence or the second heterologous polypeptide coding sequence is a chimeric heterologous polypeptide coding sequence comprising heterologous exon coding sequences interspersed with artificial host intron sequences optimized for expression in the host. In other embodiments, each of the first and second heterologous polypeptide coding sequences is individually a chimeric heterologous polypeptide coding sequence comprising heterologous exon coding sequences interspersed with artificial host intron sequences optimized for expression in the host. In certain embodiments, at least one of the first heterologous polypeptide coding sequence or the second heterologous polypeptide coding sequence replaced an entire host gene ortholog at a native locus. In certain other embodiments, each of the first and second heterologous polypeptide coding sequences individually replace an entire host gene ortholog at a native locus. In embodiments, a host animal ortholog gene corresponding to the first heterologous polypeptide coding sequence and/or the second heterologous polypeptide coding sequence has been knocked-out. In embodiments, the first and second heterologous polypeptide coding sequences of the system comprise human exon coding sequences. In certain embodiments, the one or more mutations corresponds to a human disease gene clinical variant. In other embodiments, the system further comprises and expresses one or more additional heterologous polypeptide coding sequence that contribute to the heterologous disease phenotype, optionally wherein the one or more additional heterologous polypeptide coding sequences comprises one or more mutations in exon coding sequences of the heterologous polypeptide coding sequence as compared to a wildtype reference sequence resulting in at least one amino acid change when the one or more additional heterologous polypeptide coding sequence is expressed in the host, or optionally wherein a host ortholog gene for each of the heterologous polypeptide coding sequences has been knocked-out. In embodiments, the host of the system comprises and expresses 2 to 15, or 3 to 15 heterologous polypeptide coding sequences. In embodiments, heterologous disease phenotype of the system is a monogenic human disease phenotype or alternatively, a polygenic human disease phenotype.

Provided herein in certain embodiments is a non-human animal humanized transgenic system for assessing a monogenic or polygenic human disease phenotype, comprising: a host animal comprising and expressing a first human polypeptide coding sequence and a second human polypeptide coding sequence, wherein the first and second human polypeptide coding sequences are integrated into the genome of the host animal, wherein at least one of the first human polypeptide coding sequence or the second human polypeptide coding sequence comprises one or more mutations in the human gene exon coding sequence as compared to a wildtype reference sequence resulting in at least one amino acid change when the first human gene or the second human gene is expressed in the host animal, and wherein expression of the first and second human polypeptide coding sequences contribute to the monogenic or polygenic human disease phenotype.

### Examples

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Presynaptic Terminus Activity

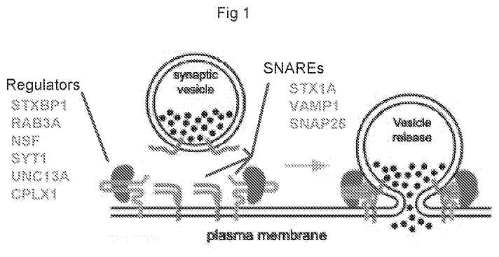

In certain embodiments, the presynaptic genes involved in neurotransmission in C. elegans are replaced with human gene sequences, specifically, the SNARE proteins, their regulators, and other proteins involved in neurotransmitter release at the presynaptic terminus. See FIG. 1. There are three SNARE proteins, syntaxin, VAMP (vesicle-associated-membrane protein) and SNAP (synaptosome-associated protein) that act to drive vesicle fusion (Malsam J, Saner T H (2011). “Organization of SNAREs within the Golgi stack”. Cold Spring Harbor Perspectives in Biology. 3 (10): a005249.). Of the neurotransmission regulators, there are six key genes acting to coordinate neurotransmitter release (STXBP1, NSF, SYT1, UNC13A, CPLX1, RAB3A). There are 31 additional genes that function at presynaptic terminus locations, and that are also involved in neurotransmitter release with involvement in human disease. As a result, there are up to 40 genes identified that may be replaced in the C. elegans with human orthologs, wherein each of those genes are useful to humanize due to their known disease associations. Creation of a humanized pathway via expression of multiple (e.g. polygenic) genes integrated into the host nematode genome creates an improved platform for disease modeling and discovery because protein-protein interactions between pairs of human genes are absolutely maintained.

In embodiments, a host nematode comprises and expresses all heterologous polypeptide coding sequences in a pathway, such as proteins and regulators (which may not necessarily be expressed) involved in neurotransmitter release at the presynaptic terminus. In other embodiments, the host nematode comprises at least two genes involved in a pathway, such as proteins and regulators involved in neurotransmitter release at the presynaptic terminus. In embodiments, the nematode may comprise from two (2) to 40 human genes, and that are expressed and contribute to the same trait or phenotype (e.g. neurotransmitter release at the presynaptic terminus). That phenotype output may be recorded using various assays known to one of skill in the art.

TABLE 1
presynaptic genes with their disease associations, C. elegans ortholog
and loss-of-function phenotypes. Human genes and their paralogs chosen
based on KEGG pathway hsa04721 for disease-associated genes:

| | | worm | | |
|---|---|---|---|---|
| human gene | disease association | gene | similarity | pheno |
| ATP6V1B1 | Renal tubular acidosis with deafness | vha-12 | 92 | lethal |
| ATP6V1B2 | congenital deafness with | vha-12 | 91 | lethal |
| | onychodystrophy, Zimmermann- | | | |
| | Laband syndrome 2 | | | |
| RAB3A | Ependymoma | rab-3 | 83 | movement |
| STX1A | Schizophrenia, Autism, Cystic fibrosis | unc-64 | 81 | lethal |
| STX1B | Generalized epilepsy with febrile | unc-64 | 81 | lethal |
| | seizures 9, | | | |
| VAMP1 | Ataxia, Myasthenia | snb-1 | 81 | lethal |
| VAMP2 | Major depressive disorder, Unipolar | snb-1 | 78 | lethal |
| | depression | | | |
| DNM1 | Early infantile epileptic encephalopathy | dyn-1 | 78 | lethal |
| | 31 | | | |
| DNM2 | Myopathy, Charcot-Marie-Tooth, | dyn-1 | 76 | lethal |
| | Lethal congenital contracture syndrome | | | |
| | 5 | | | |
| STXBP1 | Early infantile epileptic encephalopathy | unc-18 | 75 | movement |
| | 4, West syndrome, Intellectual | | | |
| | disability, Neurodevelopmental | | | |
| | disorders. Schizophrenia | | | |
| STX3 | Microvillus inclusion disease, | unc-64 | 77 | lethal |
| | Intellectual disability | | | |
| STX2 | Male sterility, Male infertility | unc-64 | 72 | lethal |
| NSF | Cocaine dependence, Epilepsy, | nsf-1 | 71 | lethal |
| | Parkinson disease | | | |
| SNAP25 | Congenital Myasthenic syndrome 18, | ric-4 | 71 | movement |
| | ADHD, Bipolar disorder, Depressive | | | |
| | disorder, Diabetes mellitus, Myasthenia | | | |
| SYT1 | Baker-Gordon syndrome, Visual | snt-1 | 71 | lethal |
| | seizure. | | | |
| SLC6A2 | Orthostatic intolerance. Mental | dat-1 | 67 | movement |
| | depression. Mitral valve prolapse | | | |
| | syndrome, Neurocirculalory asthenia. | | | |
| | Irritable heart. Depressive disorder | | | |
| SLC17A8 | Deafness, autosomal dominant 25 | eat-4 | 66 | movement |
| ATP6V0A4 | Distal renal tubular acidosis | unc-32 | 66 | lethal |
| SNAP23 | Liver Cirrhosis, Myocardial Ischemia | ric-4 | 63 | lethal |
| CASK | FG syndrome 4, Mental retardation and | lin-2 | 63 | development |
| | microcephaly, Intellectual disability | | | |
| ATP6V0A2 | Cutis laxa type IIA, Wrinkly skin | unc-32 | 62 | lethal |
| | syndrome | | | |
| CADPS | Glaucoma | unc-31 | 61 | movement |
| SYNJ1 | Early infantile epileptic | unc-26 | 60 | lethal |
| | encephalopathy-63, Parkinson disease | | | |
| | 20, Intellectual disability | | | |
| SLC18A3 | Congenital myasthenia 21 Asthma | unc-17 | 60 | lethal |
| DNAJC5 | Neuronal ceroid lipofuscinosis 4, | dnj-14 | 58 | movement |
| | Ataxia | | | |
| CPLX1 | Early infantile epileptic encephalopathy | cpx-1 | 56 | movement |
| | 63, | | | |
| TCIRG1 | Osteopetrosis 1 | unc-32 | 56 | lethal |
| UNC13A | Amyotrophic lateral sclerosis, | unc-13 | 54 | movement |
| | Intellectual disability | | | |
| CACNA1A | Early infantile epileptic encephalopathy | unc-2 | 52 | movement |
| | 42, Episodic ataxia Familial hemiplegic | | | |
| | migraine 1 , Spinocerebellar ataxia 6 | | | |
| DMXL2 | Autosomal dominant deafness 71, | rbc-1 | 50 | n.d. |
| | Polyendocrine-polyneuropathy | | | |
| | syndrome. Intellectual Disability | | | |
| EPN1 | Middle cerebral artery infarction | epn-1 | 50 | lethal |
| SNAPAP | Abnormality of brain morphology | snpn-1 | 50 | development |
| SYNGR1 | Schizophrenia, Bipolar disorder, Acute | sng-1 | 47 | movement |
| | myeloid leukemia, Libman-S acks | | | |
| | disease, Systemic lupus erythematosus | | | |
| SYN1 | X-linked epilepsy, Schizophrenia, | snn-1 | 47 | movement |
| | Depressive disorder, Autism, | | | |
| | Intellectual disability | | | |
| APBA1 | Intelligence | lin-10 | 47 | morphology |
| STXBP6 | Autism | sec-3 | 45 | lethal |
| NRXN1 | Pitt-Hopkins-like syndrome 2, | nrx-1 | 44 | development |
| | Schizophrenia | | | |
| SYP | X-linked mental retardation 96 | sph-1 | 44 | n.d. |
| BINI | Centronuclear myopathy 2 | amph-1 | 44 | morphology |
| RPH3A | Tetralogy of Fallot | rbf-1 | 42 | movement |
| BLOC1S6 | Hermansky-pudlak syndrome 9, | glo-2 | 42 | lethal |
| SV2A | Schizophrenia | svop-1 | 40 | morphology |
| RIMS1 | Cone-rod dystrophy 7 | unc-10 | 37 | movement |
| PCLO | Pontocerebellar hypoplasia 3 | unc-10 | 33 | movement |
| BSN | Heart disease, Epilepsy | cla-1 | 30 | movement |

Creation of a humanized presynaptic terminus in C. elegans involves creating clusters of humanized genes starting with the core synaptic-vesicle-fusion machinery. Genes selected for core machinery with disease associations include members of the SNARE complex (STX1A, STX1B, STX2, STX3, VAMP1, VAMP2, SNAP25 and SNAP23) Although many combinations of disease-associated SNARE are possible, in this example, the unc-64 gene in C. elegans is replaced with human STX1A, the ric-4 gene in C. elegans is replaced with human SNAP25, and the snb-1 gene in C. elegans is replaced with human VAMP1. A synthetic sequence is obtained containing the human gene coding sequence codon optimized for C. elegans. In addition, at least one but typically 3 artificial introns are inserted within the coding sequence as selected from table 2. The artificial intron sequences are derived from highly expressed nematode proteins, wherein the gene to be inserted is a chimeric comprising the human or heterologous exon coding sequences interspersed with nematode artificial intron sequences. Due to the creation of the chimeric sequence, aberrant donor and acceptor splice sites may be introduced and must be removed. The optimized chimeric heterologous sequence is inserted into the native locus using published CRISPR-transgenesis techniques (Dickinson D J and Goldstein B “CRISPR-Based Methods for Caenorhabditis elegans Genome Engineering” Genetics. 2016 March; 202(3): 885-901), wherein the nematode ortholog is replaced with the chimeric heterologous polypeptide coding sequence. Each polygenic animal is made by consecutively installing each human gene into the previously modified animal.

TABLE 2
Six artificial intron sequences
derived from nematode genes

| name | sequence |
|---|---|
| Syntron1 | Gtacttgagatccttaaacgcagtcgaaaattggtaattt |
| | tacag |
| | (SEQ ID NO: 1) |
| Syntron2 | Gtaagttcctccactagaaatatcaggtgctataattgtg |
| | ttcag |
| | (SEQ ID NO: 2) |
| Syntron3 | Gtgagttattataatttttttgatcacaacgattatttta |
| | attttcag |
| | (SEQ ID NO: 3) |
| Syntron4 | Gtgagtgattttaaacattatctgtacttaaattataaat |
| | tctctattcag |
| | (SEQ ID NO: 4) |
| Syntron5 | Gtaaataattatacattcgatgataaatttatgcgtacta |
| | tttttcag |
| | (SEQ ID NO: 5) |
| Syntron6 | Gttaaatgtacaaacaactatttgaaagattttctcaccc |
| | gattttttcag |
| | (SEQ ID NO: 6) |

Further humanization of the presynaptic terminus is performed to introduce key regulators of SNARE activity. Building on the SNARE humanized animal, the unc-18 gene is replaced with STXBP1. Similar human gene optimization and genomic insertion as used for SNARE protein insertion, a consecutive gene swap insertion procedure is used to insert the remaining regulators. The nsf-1 gene is replaced with human NSF, the snt-1 gene is replaced with human SYT1, the unc-13 gene is replaced with human UNC13A, the cpx-1 gene is replaced with human CPXL1 and the rab-3 gene is replaced with human RAB3A. The transgenic nematode comprises a humanized presynaptic terminus that uses human genes to control neurotransmission activity.

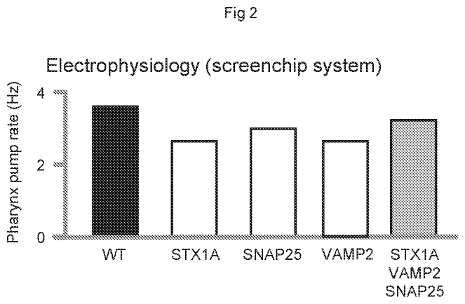

Successful installation of the humanized presynaptic terminus in the host nematode is detected by using a set of functional tests for measuring the phenotypic consequence of the polygenic gene-swap. A Screenchip electrophysiology test (U.S. Pat. No. 9,723,817) is used to determine if the heterologous polygenic animal can retain wild type electrical activity. See FIG. 2. Preparation of an animal with co-expression of human STX1A, VAMP2, and SNAP25 is shown to retain electrical activity. As shown in FIG. 2, a nematode comprising and expressing a single heterologous polypeptide coding sequence (replacing the nematode ortholog) can be useful when it rescues activity, but multiple heterologous polypeptide coding sequences that are expressed provide a polygenic system that has even greater capacity to rescue function. Similar results are expected to occur when the vesicle release regulators are installed.

The humanized polygenic pathway may be characterized utilizing additional phenotypic behavior assays such as thrashing in liquid, chemotaxis to food source, and movement on solid surface.

### Example 2: Homologous Recombination Activity

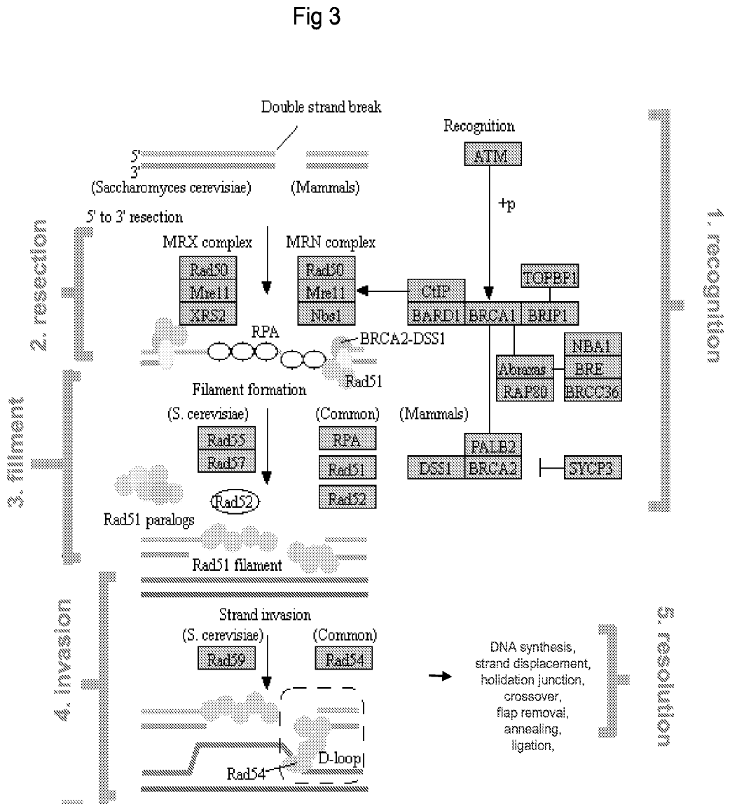

In certain embodiments provided herein is a host nematode comprising and expressing a first heterologous polypeptide coding sequence and a second heterologous polypeptide coding sequence. In embodiments, the heterologous polypeptide coding sequences are human and involved in the homologous recombination repair pathway. There are 5 steps/functionalities in executing homologous recombination repair: recognition, resection, filament, invasion, and resolution. See FIG. 3. Each involves a specific protein complex formation. (Lange S S, Takata K, Wood R D Nat Rev Cancer. 2011 Feb.; 11(2):96-110. doi: 10.1038/nrc2998). At a dsDNA break, ATM recognizes damage and recruits other recognition partners: RBBP8, BARD1, BRCA1 and BRIP1. Next the resection activity is activated and executed by RAD50, MRE11A and NBN. Filament formation occurs with RPA associations RAD51 paralogs. Strand invasion involves RAD54 activity. Resolution utilizes POLD1 with contribution from BLM, TOP3A and MUS81

TABLE 3
Homologous recombination pathway genes with their disease associations,
C. elegans ortholog and loss-of-function phenotypes. Human genes and their
paralogs chosen based on KEGG pathway hsa03440 for disease-associated genes

| | | worm | | |
|---|---|---|---|---|
| human gene | disease association | gene | similarity | pheno |
| RAD51 | Fanconi anemia complementation group R, | rad-51 | 74 | lethal |
| | Mirror movements 2, Breast cancer | | | |
| | susceptibility | | | |
| RAD54L | Somatic colonic adenocarcinoma, non- | rad-54 | 67 | lethal |
| | Hodgkin Lymphoma, non-Hodgkin, Invasive | | | |
| | ductal breast cancer | | | |
| POLDI | Mandibular hypoplasia, Deafness, | F10C2.4 | 66 | lethal |
| | Progeroid, Lipodystrophy, Colorectal cancer | | | |
| | susceptibility 10 | | | |
| TOP3A | Progressive external ophthalmoplegia with | top-3 | 57 | lethal |
| | mitochondrial DNA deletions, | | | |
| | Microcephaly, Growth restriction | | | |
| MRE11A | Ataxia-telangiectasia-like disorder 1 | mre-11 | 54 | development |
| RPA1 | Chloracne | rpa-1 | 49 | development |
| BLM | Bloom syndrome | him-6 | 49 | development |
| RAD50 | Nijmegen breakage syndrome-like disorder | rad-50 | 46 | lethal |
| RAD51D | Breast-ovarian cancer susceptibility 4 | rfs-1 | 46 | development |
| BRIP1 | Fanconi anemia complementation | dog-1 | 45 | development |
| | group J, Breast cancer early-onset | | | |
| | susceptibility | | | |
| BRCA1 | Fanconi anemia, Familial breast-ovarian | brc-1 | 39 | development |
| | cancer 1, Pancreatic cancer 4 | | | |
| NBN | Aplastic anemia, Acute lymphoblastic | ttn-1 | 38 | lethal |
| | Leukemia, Nijmegen breakage syndrome | | | |
| MUS81 | Arterial tortuosity syndrome, Emphysema, | mus-18 | 37 | development |
| | Marfan syndrome | | | |
| BARD1 | Malignant neoplasm of breast, Breast cancer | brd-1 | 36 | development |
| | susceptibility | | | |
| ATM | Ataxia-telangiectasia, B-cell non-Hodgkin | atm-1 | 35 | development |
| | lymphoma, Mantle cell lymphoma, T-cell | | | |
| | prolymphocytic leukemia, Breast cancer | | | |
| | susceptibility | | | |
| RBBP8 | Jawad syndrome, Pancreatic carcinoma, | com-1 | 35 | lethal |
| | Seckel syndrome 2 | | | |
| RAD52 | Malignant neoplasm of lung, Squamous cell | D1081.7 | 34 | movement |
| | carcinoma | | | |

Construction of the polygenic animal, comprising at least a first heterologous polypeptide coding sequence and a second heterologous polypeptide coding sequence, wherein the host animal expresses heterologous polypeptide coding sequences involved in homologous recombination repair. Replacing host nematode orthologs with heterologous polypeptide coding sequences in the homologous recombination pathway to create a humanized recognition complex involves making substitutions of ATM with atm-1, RBBP8 with com-1, BARD1 with brd-1, BRCA1 with brc-1, and BRIP1 with dog-1. For the resection system, substitutions are RAD50 with rad-50, MRE11A with mre-11 and NBN with ttn-1. For the filament formation, substitutions are RPA1 with rpa-1 and RAD51 with rad-51 and RAD52 with 1081.7 and RAD51D with rfs-1. For the strand invasion system, substitution is RAD54L with rad-54. In the resolution system, substitutions are POLD1 with F10C2.4, BLM with him-6, TOP3A with top-3 and MUS81 with mus-81. As disclosed in Example 1, construction involves creation of human chimeric gene optimized for expression in a nematode, wherein the chimeric sequence replaces the host nematode ortholog using CRISPR techniques.

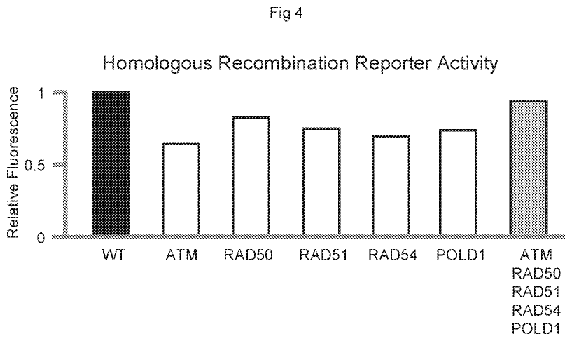

Successful humanized homologous recombination activity is measured using either an epi-chromosomal or genome integrated fluorescent reporter of HDR activity as disclosed in WO patent application PCT/US2019/45374 filed 6 Aug. 2019. As each nematode host gene is replaced with human transgene, the fluorescence activity of the reporter is measured and quantified relative to the wild type animal. See FIG. 4.

### Example 3. Modeling Variants that Modify the Severity of Disease Presentation

In one embodiment, the native C. elegans gene mthf-1 is replaced with the human coding sequence for MTHFR. Function of the MTHFR in the C. elegans background is determined by monitoring the expression of acdh-1 or growth rate. A known risk factor variant of A222V is introduced into the MTHFR sequence in the line. This strain is then used as a background for other humanizations and variant modeling. Humanizations are for epilepsy genes such as STXBP1, SCN1A, KCNQ2, CDKL5, SCN2A, PCDH19, STXBP1, PRRT2, SLC2A1, MECP2, SCN8A, UBE3, ATSC2, GABRG2, GRIN2A, FOXG1, TPP1, and GABRAL Variants in these epilepsy genes are assessed with and without the MTHFR risk factor variant A222V to see if the epilepsy gene variant has a more severe phenotype with the risk factor variant present.

### Example 4. Exemplary Digenic-Humanized Nematode

An exemplary digenic-humanized nematode was made and found to be functional. First, a monogenic-humanized animal (hSTXBP1) was constructed STXBP1 coding sequence as gene replacement of the coding sequence at the unc-18 genetic locus. This line was compared with the unc-18 KO line to confirm functional rescue.

Second, another monogenic-humanized animal (hSTX1A) was constructed expressing STX1A coding sequence as gene replacement of the coding sequence at the unc-64 genetic locus. This line was compared with the unc-64 KO line to confirm functional rescue.

Sequential construction was used to create a digenic-humanized animal (hSTXBP1; hSTX1A). Examination of the activity of the monogenic vs diagenic showed no detectable compromise of activity occurs in digenic humanized animals. This successful creation of a digenic humanized animal forecasts that further humanization of the nematode nervous system can be pursued to enable creation of a human avatar system for use in genetic diagnosis and drug discovery.

Construction of the hSTXBP1 and comparison with the unc-18 KO line was described previously. Construction of the monogenic-humanized hSTXBP1 was performed as described in Example 1 of U.S. Ser. No. 16/281,988 the contents of that Example herein is incorporated by reference.

The full deletion of unc-64 was created using guide RNAs targeting Cas9 for genomic DNA cleavage at the beginning and end of the unc-64 locus (sgRNA targeting sequences: ACAACAACATGACTAAGGAC (SEQ ID NO:7) and GAAACTTTCAGAATGCAGGA (SEQ ID NO: 8)). A gene editing mixture of Cas9 protein, guide RNAs and donor homology (5 ug Cas9, 50 pmol each sgRNA, and 500 ng donor homology) was made and microinjected into the gonad of young N2 adult hermaphrodites. Also included in the injection mix was the dpy-10 co-CRISPR selection components. Donor homology was an oligonucleotide DNA (ODN) sequence containing a right and left homology arm sequences of 35 bp lengths. In between the homology arms a cargo sequence a 3-frame start, a sequence for PCR, and a restriction enzyme site. The sequence of the ODN was:

(SEQ ID NO: 9)
CGAGACCTGTCAACAGGAACAACAACATGACTAAGTAAATAAATAAACC
CCAGAAGTCCTCCAGTCCCTCGAGGGAAGGGTTCCCATGCACTTGGTCG
ATTTGCACCT.

After injection of the gene editing mixture, 39 F1 animals containing the co-CRISPR screening phenotype were isolated to new plates. After the F2 population was established, the F1 animals were harvested and screened by PCR for the presence of the deletion. The PCR is specifically designed to distinguish between homozygous mutant, homozygous wild-type and heterozygous animals. F2 progeny from F1 animals, PCR positive as heterozygous for the deletion were isolated to try and identify homozygous animals. Four rounds of homozygosing were attempted, before it was determined that the deletion was homozygous lethal. The deletion was confirmed by DNA sequencing.

Construction of the monogenic-humanized hSTX1A occurred similarly to the construction of hSTXBP1. Guide RNAs targeting Cas9 for genomic DNA cleavage at the beginning and end of the unc-64 locus were prepared (targeting sequences: ACAACAACATGACTAAGGAC (SEQ ID NO:7) and TAATCGGCTTCGTTTCTCTG (SEQ ID NO. 8)). A gene editing mixture of Cas9 plasmid, guide RNA plasmids and donor homology plasmids (50 ng/ul Cas9, 25 ng/ul each sgRNA, and 50 ng/ul donor homology) along with selection markers was made and microinjected into the gonad of young N2 adult hermaphrodites. Donor homology was a plasmid containing a right and left homology arm sequences of 725 bp and 818 bp lengths respectively. In between the homology arms a cargo sequence encoding a nematode-codon-biased cDNA sequence for the most abundant isoform of the human STX1A gene. Immediately after the hSTX1A cDNA stop codon is a 3′UTR of the eft-3 gene. After the UTR is a selection marker cassette coding for hygromycin resistance. Three days after injection of the gene-editing reagents, Hygromycin B was added to the plates containing the progeny of the injected young adults. After 10 days, the plates were examined for surviving animals which were singled onto fresh growth plates. After progeny were established, the founding adult was harvested for PCR analysis. Allele specific PCR for desired edit was used to detect presence of desired edit. Confirmation of homozygosity was confirmed with allele-specific PCR for wild type locus. The hSTX1A strain was considered to rescue the function of the native unc-64 due to comparison to the KO of the unc-64. No homozygous unc-64 KO were isolated, which indicated that the unc-64 KO was lethal. However, homozygous KI strains with the hSTX1A replacing the unc-64 gene were isolated, indicating the function of unc-64 could be replaced by hSTX1A.

Construction of digenic-humanized animals occurred by injection of the hSTXBP1 strain with the components to create the hSTX1A strain. Homozygous animals were isolated as described above.

An alternative method to create the digenic nematode is to perform a genetic cross. Heat shock is performed on the hSTX1A plates to create males. The males of hSTX1A strain are mated with the hSTXBP1 hermaphrodites. F1 progeny are isolated on new growth plates. After F2 progeny are established, the founding F1 adult is harvested for PCR analysis. Allele-specific PCR was used to detect the presence of hSTX1A edit. F2 progeny are isolated on new growth plates. After F3 progeny are established, the founding F2 adult is harvested for PCR analysis. Allele-specific PCR is used to detect presence of hSTXBP1 and hSTX1A edits and a second allele-specific PCR is used to detect the presence of wild type (unc-18 and unc-64) at the hSTXBP1 and hSTX1A edit sites. Animals isolated as positive for hSTXBP1 and hSTX1A alleles and negative for wild-type are designated to be the desired digenic-humanized strains.

Knock-ins for the digenic-humanized and monogenic humanized animals were compared to gene knock-outs for the unc-18 and unc-64 locus (Table 4). Both the di-genic and monogenic humanized knock-ins had near wild-type activity, while the gene knock out for unc-18 was severely uncoordinated and the gene knock-out for unc-64 was not viable as homozygote.

TABLE 4

| | | | hSTXBP1; | | |
|---|---|---|---|---|---|
| Wild type | hSTXBP1 | hSTX1A | hSTX1A | unc-18 | unc-64 |
| (N2) | knock-in | knock-in | knock-in | knock-out | knock-out |
| ++++ | +++ | +++ | +++ | + | (lethal) |

### Example 5. Transgenic Nematodes Expressing Human Variants

CRISPR, crossing, self-fertilization, and similar techniques are used to create animal strains expressing multiple interacting human proteins within the synaptic bouton. Since the STXBP1 single-locus humanization line and STX1A single-locus humanization lines have already been created and crossed to generate a double-locus humanization line (as described above), humanized SNAP25 lines are created.

To generate the humanized SNAP25 line, the C. elegans ortholog ric-4 (53% identity) is replaced on Chromosome V. The human cDNA of 618 bp is optimized for expression in C. elegans and cloned into a plasmid for CRISPR/Cas9 gene editing. This plasmid also contains homology arms for ric-4 and a selection marker. A determination of whether the SNAP25 is functional is made by comparing it with the loss of function mutant which is reported to be sluggish, small, uncoordinated, and resistant to aldicarb. The donor homology plasmid is combined with the human STX1A with plasmids for the sgRNAs, Cas9, and other injection markers. All created lines are confirmed with PCR and/or sequencing, and expression levels quantified relative to the native gene by qPCR. The humanized SNAP25 line is then crossed with the STXBP1/STX1A double insertion line to create a triple insertion line, confirmed by PCR assays and sequencing. By these methods, a transgenic animal strain is created with at least three interacting human proteins replacing native orthologous proteins.

### Example 6. Molecular Phenotyping

C. elegans animals with loss of function mutations in ric-4, unc-18, and unc-64 are characterized for differential expression by RNA-seq relative to the humanized lines. Pathway reporter genes common to the three genes being manipulated are targeted. Candidates are validated by qPCR assays and those with at least a 2-fold change in expression will be selected to create fluorescent biosensors. See U.S. Pat. No. 8,937,213, herein incorporated by reference, which disclose use of inducible and constitutive promoters operably linked to reporter genes. Plasmid constructs are created as promoter-RFP fusions. Promoter regions for the candidate reporter genes are selected using ChIP-seq data from the wormbase database. Typically a 1000-2000 bp region upstream of a gene's start codon is chosen for PCR amplification and then inserted into a red fluorescent protein (RFP) expression cassette plasmid (“response plasmid”). Promoter-RFP fusion constructs (response plasmid) are co-injected with a constitutively expressed reporter plasmid (“control plasmid”) to enable ratiometric analysis. The CO2F5.3 gene is chosen for control plasmid construction because the gene has a sufficient expression (FPKM: 94) and an interstrain analysis indicates the gene has less than 6% variance across all animal types (N2 vs. CL2355 vs. BR5270 vs. UM0001). For the CO2F5.3 control plasmid, the promoter fusion is made to green fluorescent protein (GFP). The constitutive GFP expression acts as internal control allowing ratiometric normalization (RFP/GFP) for expression changes observed with each response plasmid. By these methods, at least three new molecular phenotypic indicators are identified and validated in knock-out vs. humanized lines.

We claim:

1. A non-human animal transgenic system for assessing a heterologous polygenic or monogenic phenotype, comprising:
a host non-human animal comprising and expressing a first heterologous polypeptide coding sequence and a second heterologous polypeptide coding sequence, wherein the first and second heterologous coding sequences are integrated into the host animal genome, and wherein expression of the first and second heterologous polypeptide coding sequences in the animal contribute to the heterologous phenotype.

2. The system of claim 1 wherein the host non-human animal is a nematode or a zebrafish.

3. The system of claim 1, wherein at least one of the first heterologous polypeptide coding sequence or the second heterologous polypeptide coding sequence is a chimeric heterologous polypeptide coding sequence comprising heterologous exon coding sequences interspersed with artificial host intron sequences optimized for expression in the host.

4. The system of claim 1, wherein each of the first and second heterologous polypeptide coding sequences is individually a chimeric heterologous polypeptide coding sequence comprising heterologous exon coding sequences interspersed with artificial host intron sequences optimized for expression in the host animal.

5. The system of claim 1, wherein at least one of the first heterologous coding sequence or the second heterologous coding sequence replaced an entire host gene ortholog at a native locus.

6. The system of claim 1, wherein each of the first and second heterologous coding sequences individually replaced an entire host gene ortholog at a native locus.

7. The system of claim 1, wherein host ortholog gene sequence corresponding to the first heterologous coding sequence and/or the second heterologous coding sequence has been knocked-out.

8. The system of claim 1, wherein the first and second heterologous coding sequences comprise human exon coding sequences.

9. The system of claim 1, wherein at least one of the first heterologous polypeptide coding sequence or the second heterologous polypeptide coding sequence comprises one or more mutations in the first and/or second heterologous polypeptide coding sequence coding sequences as compared to a wildtype reference sequence resulting in at least one amino acid change in the first and/or second polypeptide coding sequences when the one or more additional heterologous polypeptide coding sequence is expressed in the host.

10. The system of claim 9, wherein the mutation corresponds to a human disease gene clinical variant.

11. The system of claim 1, further comprising and expressing one or more additional heterologous polypeptide coding sequence that contributes to the heterologous phenotype.

12. The system of claim 11, wherein the one or more additional heterologous polypeptide coding sequences comprises one or more mutations in polypeptide coding sequence as compared to a wildtype reference sequence resulting in at least one amino acid change when the one or more additional heterologous polypeptide coding sequence is expressed in the host.

13. The system of claim 11, wherein the host animal comprises and expresses 3 to 15 heterologous polypeptide coding sequences.

14. The system of claim 13, wherein a host ortholog gene corresponding to each of the heterologous polypeptide coding sequences has been knocked-out.

15. The system of claim 1, wherein the heterologous phenotype is a monogenic human disease phenotype.

16. The system of claim 1, wherein the heterologous phenotype is a polygenic human disease phenotype.

17. A non-human animal transgenic system for assessing a heterologous disease phenotype, comprising:
a host animal comprising and expressing a first heterologous polypeptide coding sequence and a second heterologous polypeptide coding sequence, wherein the first and second heterologous polypeptide coding sequences are integrated into the host genome, wherein at least one of the first heterologous polypeptide coding sequence or the second heterologous polypeptide coding sequence comprises one or more mutations in the heterologous polypeptide coding sequence as compared to a wildtype reference sequence resulting in at least one amino acid change when the first heterologous polypeptide coding sequence or the second heterologous polypeptide coding sequence is expressed,
and wherein expression of the first and second heterologous polypeptide coding sequence contribute to the heterologous disease phenotype.

18. The system of claim 17, wherein at least one of the first heterologous polypeptide coding sequence or the second heterologous polypeptide coding sequence is a chimeric heterologous polypeptide coding sequence comprising heterologous exon coding sequences interspersed with artificial host intron sequences optimized for expression in the host.

19. The system of claim 17, wherein each of the first and second heterologous polypeptide coding sequences is individually a chimeric heterologous polypeptide coding sequence comprising heterologous exon coding sequences interspersed with artificial host intron sequences optimized for expression in the host.

20. The system of claim 17, wherein at least one of the first heterologous polypeptide coding sequence or the second heterologous polypeptide coding sequence replaced an entire host gene ortholog at a native locus.

21. The system of claim 17, wherein each of the first and second heterologous polypeptide coding sequences individually replace an entire host gene ortholog at a native locus.

22. The system of claim 17, wherein a host animal ortholog gene corresponding to the first heterologous polypeptide coding sequence and/or the second heterologous polypeptide coding sequence has been knocked-out.

23. The system of claim 17, wherein the first and second heterologous polypeptide coding sequences comprise human exon coding sequences.

24. The system of claim 17, wherein the one or more mutations corresponds to a human disease gene clinical variant.

25. The system of claim 17, further comprising and expressing one or more additional heterologous polypeptide coding sequence that contribute to the heterologous disease phenotype.

26. The system of claim 25, wherein the one or more additional heterologous polypeptide coding sequences comprises one or more mutations in exon coding sequences of the heterologous polypeptide coding sequence as compared to a wildtype reference sequence resulting in at least one amino acid change when the one or more additional heterologous polypeptide coding sequence(s) is expressed in the host.

27. The system of claim 25, wherein the host comprises and expresses 3 to 15 heterologous polypeptide coding sequences.

28. The system of claim 25, wherein a host ortholog gene for each of the heterologous polypeptide coding sequences has been knocked-out.

29. The system of claim 17, wherein the heterologous disease phenotype is a monogenic human disease phenotype.

30. The system of claim 17, wherein the heterologous disease phenotype is a polygenic human disease phenotype.

31. A non-human animal humanized transgenic system for assessing a monogenic or polygenic human disease phenotype, comprising:
a host animal comprising and expressing a first human polypeptide coding sequence and a second human polypeptide coding sequence, wherein the first and second human polypeptide coding sequences are integrated into the genome of the host animal, wherein at least one of the first human polypeptide coding sequence or the second human polypeptide coding sequence comprises one or more mutations in the human gene exon coding sequence as compared to a wildtype reference sequence resulting in at least one amino acid change when the first human gene or the second human gene is expressed in the host animal,
and wherein expression of the first and second human polypeptide coding sequences contribute to the monogenic or polygenic human disease phenotype.

32. The system of claim 31, wherein at least one of the first heterologous polypeptide coding sequence or the second heterologous polypeptide coding sequence is a chimeric heterologous polypeptide coding sequence comprising heterologous exon coding sequences interspersed with artificial host nematode intron sequences optimized for expression in the host animal.

33. The system of claim 31, wherein each of the first and second heterologous polypeptide coding sequence is individually a chimeric heterologous polypeptide coding sequence comprising heterologous exon coding sequences interspersed with artificial host intron sequences optimized for expression in the host animal.

34. The system of claim 31, wherein at least one of the first heterologous polypeptide coding sequence or the second heterologous polypeptide coding sequence replaced an entire host animal gene ortholog at a native locus.

35. The system of claim 31, wherein each of the first and second heterologous polypeptide coding sequences individually replace an entire host nematode gene ortholog at a native locus.

36. The system of claim 31, wherein a host nematode ortholog gene of the first heterologous polypeptide coding sequence and/or the second heterologous polypeptide coding sequence has been knocked-out.

37. The system of claim 31, wherein the one or more mutations corresponds to a human disease gene clinical variant.

38. The system of claim 31, further comprising and expressing one or more additional heterologous polypeptide coding sequences that contribute to the monogenic or polygenic human disease phenotype.

39. The system of claim 38, wherein the one or more additional heterologous polypeptide coding sequences comprise one or more mutations in exon coding sequences of the heterologous polypeptide coding sequence as compared to a wildtype reference sequence resulting in at least one amino acid change when the one or more additional heterologous polypeptide coding sequence is expressed in the host animal.

40. The system of claim 38, wherein the host comprises and expresses 3 to 15 polypeptide coding sequences.

41. The system of claim 38, wherein a host ortholog gene corresponding to each of the heterologous polypeptide coding sequences has been knocked-out.

42. The system of claim 31, wherein the phenotype is a monogenic human disease phenotype.

43. The system of claim 31, wherein the phenotype is a polygenic human disease phenotype.
